(19) United States
(12) Patent Application Publication
(10) Pub. No.: US 2014/0001056 A1
(43) Pub. Date: Jan. 2, 2014

(54) METHOD USING FLUORINATED AMPHIPHILES

(71) Applicants: John Hagan Pryce Bayley, Oxford (GB); Pinky Raychaudhuri, Oxford (GB)

(72) Inventors: John Hagan Pryce Bayley, Oxford (GB); Pinky Raychaudhuri, Oxford (GB)

(73) Assignee: ISIS INNOVATION LIMITED, Oxford (GB)

(21) Appl. No.: 13/979,584

(22) PCT Filed: Jan. 11, 2012
(86) PCT No.: PCT/GB12/50048

§ 371 Date: Sep. 19, 2013

(30) Foreign Application Priority Data
Jan. 12, 2011 (GB) 1100516.2

(51) Int. Cl. G01N 33/487

(52) U.S. Cl. CPC G01N 33/48721
USPC 205/778; 564/15; 536/18.4; 252/408.1; 205/793; 205/780.5; 204/403.06

(57) ABSTRACT

The invention relates to a method of inhibiting the insertion of one or more membrane proteins into a lipid bilayer. The invention also relates to a method of inserting a pre-determined number of membrane proteins into a lipid bilayer and lipid bilayers having a pre-determined number of membrane proteins inserted therein. The lipid bilayers of the invention are useful as sensor arrays, particularly for sequencing nucleic acids.

### FIELD OF THE INVENTION

The invention relates to a method of inhibiting the insertion of one or more membrane proteins into a lipid bilayer. The invention also relates to a method of inserting a pre-determined number of membrane proteins into a lipid bilayer and lipid bilayers having a pre-determined number of membrane proteins inserted therein. The lipid bilayers of the invention are useful in sensor arrays, particularly for sequencing nucleic acids.

### BACKGROUND OF THE INVENTION

A fluorinated amphiphile (F-amphiphile) comprises a polar head group and a hydrophobic tail that features a partially or largely fluorinated chain. Because fluorocarbons do not mix with the hydrocarbon chains of common bilayer lipids, F-amphiphiles do not in general solubilize membranes (Chabaud et al. (1998) Biochimie 80, 515-530). For example, F-amphiphiles enhance the insertion of diphtheria toxin T-domain into preformed lipid bilayers by reducing nonproductive aggregation of the protein (Palchevskyy et al. (2006) Biochemistry 45, 2629-2635). Other work has established that F-amphiphiles are compatible with the in vitro protein synthesis, folding and oligomerization of the pentameric mechanosensitive channel MscL (Park et al. (2007) Biochem J 403, 183-187). The same report also suggested that F-amphiphiles facilitate direct and rapid incorporation of functional MscL into preformed lipid bilayers. Furthermore, non-ionic or zwitterionic F-amphiphiles are considered less denaturing for proteins than their hydrocarbon-based counterparts (Breyton et al. (2004) FEBS Letters 564, 312-318). Previous reports have also suggested that some fluorinated amphiphiles, although unable to solubilize membranes, maintain the solubility of integral membrane proteins following transfer from hydrogenated surfactants (Breyton et al. supra).

Stochastic sensors can be created by placing a single pore of nanometer dimensions in an insulating membrane and measuring voltage-driven ionic transport through the pore in the presence of analyte molecules. The frequency of occurrence of fluctuations in the current reveals the concentration of an analyte that binds within the pore. The identity of an analyte is revealed through its distinctive current signature, notably the duration and extent of current block (Braha, O., Walker, B., Cheley, S., Kasianowicz, J. J., Song, L., Gouaux, J. E., and Bayley, H. (1997) Chem. Biol. 4, 497-505; and Bayley, H., and Cremer, P. S. (2001) Nature 413, 226-230).

Engineered versions of the bacterial pore forming toxin α-hemolysin (α-HL) have been used for stochastic sensing of many classes of molecules (Bayley, H., and Cremer, P. S. (2001) Nature 413, 226-230; Shin, S., H., Luchian, T., Cheley, S., Braha, O., and Bayley, H. (2002) Angew. Chem. Int. Ed. 41, 3707-3709; and Guan, X., Gu, L.-Q., Cheley, S., Braha, O., and Bayley, H. (2005) Chem Bio Chem 6, 1875-1881). In the course of these studies, it was found that attempts to engineer α-HL to bind small organic analytes directly can prove taxing, with rare examples of success (Guan, X., Gu, L.-Q., Cheley, S., Braha, O., and Bayley, H. (2005) Chem Bio Chem 6, 1875-1881). Fortunately, a different strategy was discovered, which utilised non-covalently attached molecular adaptors, notably cyclodextrins (Gu, L.-Q., Braha, O., Conlan, S., Cheley, S., and Bayley, H. (1999) Nature 398, 686-690), but also cyclic peptides (Sanchez-Quesada, J., Ghadiri, M. R., Bayley, H., and Braha, O. (2000) J. Am. Chem. Soc. 122, 11758-11766) and cucurbiturils (Braha, O., Webb, J., Gu, L.-Q., Kim, K., and Bayley, H. (2005) Chem Phys Chem 6, 889-892). Cyclodextrins become transiently lodged in the α-HL pore and produce a substantial but incomplete channel block. Organic analytes, which bind within the hydrophobic interiors of cyclodextrins, augment this block allowing analyte detection (Gu, L.-Q., Braha, O., Conlan, S., Cheley, S., and Bayley, H. (1999) Nature 398, 686-690).

There is currently a need for rapid and cheap DNA or RNA sequencing technologies across a wide range of applications. Existing technologies are slow and expensive mainly because they rely on amplification techniques to produce large volumes of nucleic acid and require a high quantity of specialist fluorescent chemicals for signal detection. Stochastic sensing has the potential to provide rapid and cheap DNA sequencing by reducing the quantity of nucleotide and reagents required.

### SUMMARY OF THE INVENTION

The inventors have extended previous work with F-amphiphiles to α-hemolysin (α-HL), which forms a 232.4 kDa heptameric protein pore (Song et al. (1996) Science 274, 1859-1865). The crystal structure of the pore reveals a 14-stranded transmembrane β barrel capped by an extramembraneous domain, which encloses a roughly spherical water-filled cavity. The inventors have also examined MspA, a 157 kDa octameric porin from Mycobacterium smegmatis, and Kcv, a 42 kDa tetrameric potassium channel, which is largely α-helical. Kcv is encoded by the chlorella virus PBCV-1 (Plugge et al. (2000) Science 287, 1641-1644).

By contrast with previous work with other membrane proteins, the inventors have surprisingly demonstrated that F-amphiphiles sequester the α-HL pore in the surfactant phase such that it is unavailable for insertion into lipid bilayers and liposomes. They have also surprisingly demonstrated that, after the insertion of α-HL pores into a lipid bilayer from a standard detergent, the addition of an F-amphiphile arrests further insertion without compromising bilayer stability or affecting the pores that have already inserted. This phenomenon provides a means to control the number of α-HL pores that insert into preformed planar bilayers and liposomes. Kcv and MspA behaved in a similar manner.

Accordingly, the invention provides a method for inhibiting the insertion of one or more membrane proteins into a lipid bilayer, comprising (a) contacting the proteins and lipid bilayer with a fluorinated amphiphile (F-amphiphile) under conditions that in the absence of the F-amphiphile allow the insertion of the proteins into the lipid bilayer and (b) thereby inhibiting the insertion of the proteins into the lipid bilayer.

The invention also provides:

a method for inserting a pre-determined number of membrane proteins into a lipid bilayer, comprising (a) contacting more than the pre-determined number of the proteins with the lipid bilayer under conditions that allow the insertion of the proteins into the lipid bilayer and (b) once the pre-determined number of membrane proteins have inserted in the lipid bilayer contacting the proteins and lipid bilayer with a F-amphiphile and thereby inhibiting further insertion of the proteins into the lipid bilayer;

a lipid bilayer having a predetermined number of membrane proteins inserted therein produced using a method of the invention;

a method of determining the presence or absence of an analyte, comprising:
(a) contacting the analyte with a lipid bilayer of the invention, which comprises a pore or an ion channel, so that the analyte interacts with the pore or channel; and
(b) measuring the current passing through the pore or channel during the interaction and thereby determining the presence or absence of the analyte;

a method of sequencing a target nucleic acid sequence, comprising:
(a) contacting the target sequence with a lipid bilayer of the invention, which comprises at least one transmembrane pore having a molecular adaptor and an exonuclease covalently attached thereto, such that the exonuclease digests an individual nucleotide from one end of the target sequence;
(b) contacting the nucleotide with the pore so that the nucleotide interacts with the adaptor;
(c) measuring the current passing through the pore during the interaction and thereby determining the identity of the nucleotide; and
(d) repeating steps (a) to (c) at the same end of the target sequence and thereby determining the sequence of the target sequence;

a method of estimating the sequence of a target nucleic acid sequence, comprising:
(a) contacting the target sequence with a lipid bilayer of the invention, which comprises at least one transmembrane pore so that the target sequence translocates through the pore and a proportion of the nucleotides in the target sequence interacts with the pore; and
(b) measuring the current passing through the pore during each interaction and thereby determining the sequence of the target sequence; and

a kit for inserting a pre-determined number of membrane proteins into a lipid bilayer comprising (a) one or more membrane proteins and (b) a fluorinated amphiphile, wherein the membrane proteins are derived from α-hemolysin (α-HL), MspA from Mycobacterium smegmatis or Kcv of chlorella virus PBCV-1.

### DESCRIPTION OF THE FIGURES

FIG. 1 shows the chemical structures of the F-amphiphiles used in the Example: (A) Fluorinated fos-choline (F₆FC), CMC: 2.2 mM; (B) Fluorinated octyl maltoside (F₆OM), CMC: 1.02 mM (43); (C) C₆F₁₃C₂H₄—S-poly[tris(hydroxymethyl)aminomethane] (F₆TAC), n ˜7 to 8 and CMC: 0.3 mM. The critical micelle concentration (CMC) values quoted here most likely do not represent the concentrations at which spherical micelles are formed, which would be the case with hydrogenated amphiphiles. F-amphiphiles probably form more elaborate aggregates above the CMC.

FIG. 2 shows dye leakage assays to determine the effects of F-amphiphiles on LUV permeabilization by α-HL. The release of self-quenched CF causes an increase in fluorescence emission at 520 nm, which is monitored in the assays. Panel A: (i) insertion of α-HL monomer in the presence of F₆FC; (ii) insertion of α-HL monomer in the presence of F₆OM; (iii) insertion of α-HL monomer in the presence of F₆TAC. Panel B: (i) insertion of α-HL heptamer in the presence of F₆FC; (ii) insertion of α-HL heptamer in the presence of F₆OM; (iii) insertion of α-HL heptamer in the presence of F₆TAC. The traces are color coded: insertion of α-HL in the absence of an F-amphiphile (black open squares); insertion of α-HL at below the CMC (red open circles); insertion of α-HL at around the CMC (blue filled triangles); insertion of α-HL at above the CMC (green open diamonds). The arrow in each trace indicates the addition of Triton X-100 to the cuvette to fully lyse the liposomes. For further details, see Table 6.

FIG. 3 shows the effects of F-amphiphiles on α-HL pore formation in planar lipid bilayers. (A) Multiple insertions of WT-α-HL heptamers (30 ng mL⁻¹, cis) in the absence of F-amphiphile at +100 mV. (B) Multiple insertions of WT-α-HL heptamers (30 ng mL⁻¹, cis) in the presence of F₆FC (10 mM, trans) at +100 mV. (C, D) Arrest of the insertion of α-HL heptamers (30 ng mL⁻¹, cis) and monomers (30 ng mL⁻¹, cis), respectively, at +100 mV, by the addition of F₆FC (arrow, 10 mM, cis). (E, F) Arrest of the insertion of α-HL heptamers (30 ng mL⁻¹, cis) and monomers (30 ng mL⁻¹, cis), respectively, at +100 mV, in the presence of F₆TAC (arrow, 2 mM, cis). All recordings were made in buffer A.

FIG. 4 shows the effect of F₆FC on the insertion of the porin MspA and the potassium channel Kcv into planar lipid bilayers. Panel (i) (A) MspA (NNNRRK mutant) pore activity in the absence of F-amphiphile at +50 mV. (B) Effect of the addition of F₆FC (arrow, 10 mM, cis) after the insertion of an MspA pore. No further insertion events occur. The recording buffer for MspA was 1.0 M KCl, 10 mM Tris.HCl, pH 7.0. Panel (ii) (A) WT-Kcv channel activity in the absence of F-amphiphile at +100 mV. (B) Condensed view of the trace from which ‘C’ was taken. Numerous individual openings are seen, with occasional coincident openings of two and three channels. (C) WT-Kcv was added to the cis chamber just after F₆FC addition (arrow, 10 mM, cis) at +100 mV. No openings are seen. The recording buffer for Kcv was 200 mM KCl, 10 mM HEPES, pH 7.0.

FIG. 5 shows the effects of F₆FC on the IV curves of α-HL, MspA and Kcv in planar bilayers. (A) IV curve of WT-α-HL heptamer in the absence (open circles) and presence (open triangles) of F₆FC (10 mM, cis). The recording buffer was buffer A. (B) IV curve of a single MspA pore in the absence (open circles) and presence (open triangles) of F₆FC (10 mM, cis). The recording buffer for MspA was 1.0 M KCl, 10 mM Tris.HCl, pH 7.0. (C) IV curve of single channel of WT-Kcv in the absence (open circles) and presence (open triangles) of F₆FC (10 mM, cis). The recording buffer was 200 mM KCl, 10 mM HEPES, pH 7.0. For all IV curves, each data point is the mean±S.D. from three separate single channel recordings.

FIG. 6 shows the effects of F₆FC on the binding of βCD to the WT-α-HL pore. (A) Representative single channel current traces from a WT-α-HL heptamer at ±40 mV showing blockades by βCD (40 μM, trans) in the presence of F₆FC (10 mM, cis). Levels 1 and 2 indicate the unoccupied and occupied α-HL pore. (B) IV curve of α-HL in the absence (open circles) and presence of bound βCD (open triangles). (C, D) Representative dwell time histograms at +40 mV for the interaction of βCD (40 μM, trans) with WT-α-HL in the presence of 10 mM F₆FC (cis): τₒₙ, inter-event interval; τ_{off}, βCD dwell time. The results from the kinetic analysis are summarized in Table 7. The recording buffer was 1 M NaCl, 10 mM Na₂HPO₄, pH 7.5.

FIG. 7 shows the titration with F₆FC (cis) to determine the concentration at which α-HL heptamer insertion is blocked. The recording buffer was buffer A. The concentration of α-HL heptamer in all cases was 30 ng mL⁻¹. The final concentrations of F₆FC were (A to F) 3 mM, 4 mM, 6 mM, 8 mM, 9 mM and 10 mM. The arrows in the traces indicate the points at which F₆FC was added.

FIG. 8 shows the stability of the α-HL pore in the presence of F-amphiphiles. (A) WT-α-HL monomer and heptamer were incubated at room temperature for 5 min with F-amphiphiles at concentrations 5 times the reported CMCs. Monomer and heptamer samples (˜0.25 μg) were then run on 12% Bis-Tris SDS polyacrylamide gels in XT-MES buffer at 200 V. Lanes 1, 5: WT-α-HL heptamer and monomer, respectively, in absence of F-amphiphiles; lanes 2, 6: WT-α-HL+heptamer and monomer, respectively, incubated with F₆FC; lanes 3, 7: WT-α-HL heptamer and monomer, respectively, incubated with F₆OM; lanes 4, 8: WT-α-HL heptamer and monomer, respectively, incubated with F₆TAC. (B) Hemolytic activity of the αHL monomer in the presence of F-amphiphiles. The concentrations of F₆FC, F₆OM and F₆TAC in lanes B1, C1 and D1 (after the addition of red cells) were 10 mM, 5 mM and 2 mM respectively. αHL prepared by IVTT (5 μL) was added to A1, B1, C1 and D1. The protein and amphiphile were then subjected to two-fold serial dilution in 10 mM MOPS, 150 mM NaCl, pH 7.4, over the remaining 11 wells (final volume 50 μL per well), followed by the addition of washed 1% rRBCs (50 μL) to each well. The concentrations of F-amphiphile in wells 1 are above the CMC. In all wells, the protein was subjected to F-amphiphile at above the CMC before dilution.

FIG. 9 shows the aggregates formed by F-amphiphiles and the proposed mechanism of protein sequestration by them. (A) TEM of isolated multilayer vesicles in a dilute dispersion (0.5% w/v) of F₈FC after gentle shaking by hand at room temperature. Bar=50 nm. F₈FC is a single-chain F-alkyl phosphocholine amphiphile with a C₈F₁₇C₂H₄— chain (cf. the C₆F₁₃C₂H₄— chain in F₆FC, FIG. 1). (B) Proposed mechanism of action in which membrane proteins partition into the aggregates formed by F-amphiphiles (dark) and are therefore unavailable for insertion into planar lipid bilayers (light).

### DESCRIPTION OF THE SEQUENCE LISTING

SEQ ID NO: 1 shows the polynucleotide sequence encoding one subunit of wild type α-hemolysin (α-HL).

SEQ ID NO: 2 shows the amino acid sequence of one subunit of wild type α-HL. Amino acids 2 to 6, 73 to 75, 207 to 209, 214 to 216 and 219 to 222 form α-helices. Amino acids 22 to 30, 35 to 44, 52 to 62, 67 to 71, 76 to 91, 98 to 103, 112 to 123, 137 to 148, 154 to 159, 165 to 172, 229 to 235, 243 to 261, 266 to 271, 285 to 286 and 291 to 293 form β-strands. All the other non-terminal amino acids, namely 7 to 21, 31 to 34, 45 to 51, 63 to 66, 72, 92 to 97, 104 to 111, 124 to 136, 149 to 153, 160 to 164, 173 to 206, 210 to 213, 217, 218, 223 to 228, 236 to 242, 262 to 265, 272 to 274 and 287 to 290 form loop regions. Amino acids 1 and 294 are terminal amino acids.

SEQ ID NO: 3 shows the polynucleotide sequence encoding one subunit of α-HL L135C/N139Q (HL-CQ).

SEQ ID NO: 4 shows the amino acid sequence of one subunit of α-HL L135C/N139Q (HL-CQ). The same amino acids that form α-helices, β-strands and loop regions in wild type α-HL form the corresponding regions in this subunit.

SEQ ID NO: 5 shows the codon optimised polynucleotide sequence derived from the sbcB gene from E. coli. It encodes the exonuclease I enzyme (EcoExo I) from E. coli.

SEQ ID NO: 6 shows the amino acid sequence of exonuclease I enzyme (EcoExo I) from E. coli. This enzyme performs processive digestion of 5′ monophosphate nucleosides from single stranded DNA (ssDNA) in a 5′ to 3′ direction. Amino acids 60 to 68, 70 to 78, 80 to 93, 107 to 119, 124 to 128, 137 to 148, 165 to 172, 182 to 211, 213 to 221, 234 to 241, 268 to 286, 313 to 324, 326 to 352, 362 to 370, 373 to 391, 401 to 454 and 457 to 475 form α-helices. Amino acids 10 to 18, 28 to 26, 47 to 50, 97 to 101, 133 to 136, 229 to 232, 243 to 251, 258 to 263, 298 to 302 and 308 to 311 form β-strands. All the other non-terminal amino acids, 19 to 27, 37 to 46, 51 to 59, 69, 79, 94 to 96102 to 106, 120 to 123, 129 to 132, 149 to 164, 173 to 181, 212, 222 to 228 233, 242, 252 to 257, 264 to 267, 287 to 297, 303 to 307, 312, 325, 353 to 361, 371, 372, 392 to 400, 455 and 456, form loops. Amino acids 1 to 9 are terminal amino acids. The overall fold of the enzyme is such that three regions combine to form a molecule with the appearance of the letter C, although residues 355-358, disordered in the crystal structure, effectively convert this C into an O-like shape. The amino terminus (1-206) forms the exonuclease domain and has homology to the DnaQ superfamily, the following residues (202-354) form an SH3-like domain and the carboxyl domain (359-475) extends the exonuclease domain to form the C-like shape of the molecule. Four acidic residues of EcoExo I are conserved with the active site residues of the DnaQ superfamily (corresponding to D15, E17, D108 and D186). It is suggested a single metal ion is bound by residues D15 and 108. Hydrolysis of DNA is likely catalyzed by attack of the scissile phosphate with an activated water molecule, with H181 being the catalytic residue and aligning the nucleotide substrate.

SEQ ID NO: 7 shows the codon optimised polynucleotide sequence derived from the xthA gene from E. coli. It encodes the exonuclease III enzyme from E. coli.

SEQ ID NO: 8 shows the amino acid sequence of the exonuclease III enzyme from E. coli. This enzyme performs distributive digestion of 5′ monophosphate nucleosides from one strand of double stranded DNA (dsDNA) in a 3′-5′ direction. Enzyme initiation on a strand requires a 5′ overhang of approximately 4 nucleotides. Amino acids 11 to 13, 15 to 25, 39 to 41, 44 to 49, 85 to 89, 121 to 139, 158 to 160, 165 to 174, 181 to 194, 198 to 202, 219 to 222, 235 to 240 and 248 to 252 form α-helices. Amino acids 2 to 7, 29 to 33, 53 to 57, 65 to 70, 75 to 78, 91 to 98, 101 to 109, 146 to 151, 195 to 197, 229 to 234 and 241 to 246 form β-strands. All the other non-terminal amino acids, 8 to 10, 26 to 28, 34 to 38, 42, 43, 50 to 52, 58 to 64, 71 to 74, 79 to 84, 90, 99, 100, 110 to 120, 140 to 145, 152 to 157, 161 to 164, 175 to 180, 203 to 218, 223 to 228, 247 and 253 to 261, form loops. Amino acids 1, 267 and 268 are terminal amino acids. The enzyme active site is formed by loop regions connecting β₁-α₁, β₃-β₄, β₅-β₆, β_{III}-α_{I}, β_{IV}-α_{II} and β_{V}-β_{VI} (consisting of amino acids 8-10, 58-64, 90, 110-120, 152-164, 175-180, 223-228 and 253-261 respectively). A single divalent metal ion is bound at residue E34 and aids nucleophilic attack on the phosphodiester bond by the D229 and H259 histidine-aspartate catalytic pair.

SEQ ID NO: 9 shows the codon optimised polynucleotide sequence derived from the recJ gene from T. thermophilus. It encodes the RecJ enzyme from T. thermophilus (TthRecJ-cd).

SEQ ID NO: 10 shows the amino acid sequence of the RecJ enzyme from T. thermophilus (TthRecJ-cd). This enzyme performs processive digestion of 5′ monophosphate nucleosides from ssDNA in a 5′-3′ direction. Enzyme initiation on a strand requires at least 4 nucleotides. Amino acids 19 to 33, 44 to 61, 80 to 89, 103 to 111, 136 to 140, 148 to 163, 169 to 183, 189 to 202, 207 to 217, 223 to 240, 242 to 252, 254 to 287, 302 to 318, 338 to 350 and 365 to 382 form α-helices. Amino acids 36 to 40, 64 to 68, 93 to 96, 116 to 120, 133 to 135, 294 to 297, 321 to 325, 328 to 332, 352 to 355 and 359 to 363 form β-strands. All the other non-terminal amino acids, 34, 35, 41 to 43, 62, 63, 69 to 79, 90 to 92, 97 to 102, 112 to 115, 121 to 132, 141 to 147, 164 to 168, 184 to 188203 to 206, 218 to 222, 241, 253, 288 to 293, 298 to 301, 319, 320, 326, 327, 333 to 337, 351 to 358 and 364, form loops. Amino acids 1 to 18 and 383 to 425 are terminal amino acids. The crystal structure has only been resolved for the core domain of RecJ from Thermus thermophilus (residues 40-463). To ensure initiation of translation and in vivo expression of the RecJ core domain a methionine residue was added at its amino terminus, this is absent from the crystal structure information. The resolved structure shows two domains, an amino (2-253) and a carboxyl (288-463) region, connected by a long α-helix (254-287). The catalytic residues (D46, D98, H122, and D183) co-ordinate a single divalent metal ion for nucleophilic attack on the phosphodiester bond. D46 and H120 proposed to be the catalytic pair; however, mutation of any of these conserved residues in the E. coli RecJ was shown to abolish activity.

SEQ ID NO: 11 shows the codon optimised polynucleotide sequence derived from the bacteriphage lambda exo (redX) gene. It encodes the bacteriophage lambda exonuclease.

SEQ ID NO: 12 shows the amino acid sequence of the bacteriophage lambda exonuclease. The sequence is one of three identical subunits that assemble into a trimer. The enzyme performs highly processive digestion of nucleotides from one strand of dsDNA, in a 5′-3′ direction (http://www.neb.com/nebecomm/products/productM0262.asp). Enzyme initiation on a strand preferentially requires a 5′ overhang of approximately 4 nucleotides with a 5′ phosphate. Amino acids 3 to 10, 14 to 16, 22 to 26, 34 to 40, 52 to 67, 75 to 95, 135 to 149, 152 to 165 and 193 to 216 form α-helices. Amino acids 100 to 101, 106 to 107, 114 to 116, 120 to 122, 127 to 131, 169 to 175 and 184 to 190 form β-strands. All the other non-terminal amino acids, 11 to 13, 17 to 21, 27 to 33, 41 to 51, 68 to 74, 96 to 99, 102 to 105, 108 to 113, 117 to 119, 123 to 126, 132 to 134, 150 to 151, 166 to 168, 176 to 183, 191 to 192, 217 to 222, form loops. Amino acids 1, 2 and 226 are terminal amino acids. Lambda exonuclease is a homo-trimer that forms a toroid with a tapered channel through the middle, apparently large enough for dsDNA to enter at one end and only ssDNA to exit at the other. The catalytic residues are undetermined but a single divalent metal ion appears bound at each subunit by residues D119, E129 and L130.

SEQ ID NO: 13 shows the polynucleotide sequence encoding one subunit of wild type MspA from Mycobacterium smegmatis.

SEQ ID NO: 14 shows the amino acid sequence of one subunit of wild type MspA from Mycobacterium smegmatis.

SEQ ID NO: 15 shows the polynucleotide sequence encoding one subunit of wild type Kcv.

SEQ ID NO: 16 shows the amino acid sequence of one subunit of wild type Kcv.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a F-amphiphile” includes “F-amphiphiles”, reference to “a membrane protein” includes two or more such proteins, reference to “a molecular adaptor” includes two or more such adaptors, and the like.

All publications, patents and patent applications cited herein, whether supra or infra, are hereby incorporated by reference in their entirety.

### Methods

The invention provides a method for inhibiting the insertion of one or more membrane proteins into a lipid bilayer. It also provides a method for controlling or limiting the number of membrane proteins which insert into a lipid bilayer. The method comprises contacting the proteins and the lipid bilayer with a fluorinated amphiphile (F-amphiphile) under conditions that in the absence of the F-amphiphile allow the insertion of the proteins into the lipid bilayer. The F-amphiphile inhibits the insertion of the protein into the lipid bilayer. Any number of membrane proteins, such as such as 1, 2, 4, 5, 7, 8, 10, 12, 14, 15, 20, 30, 40, 50, 100 or more proteins, may be used. The one or more membrane proteins may be an oligomeric protein, such as a transmembrane pore or ion channel, or one or more monomeric proteins, such as one or more transmembrane pore monomers or ion channel monomers.

The invention also concerns using a F-amphiphile to insert a predetermined or controlled number of membrane proteins into a lipid bilayer. In this embodiment, the method comprises contacting more than the predetermined number of proteins with the lipid bilayer under conditions that allow the insertion of the proteins into the lipid bilayer. Once the pre-determined number of proteins have inserted in the lipid bilayer, the proteins and lipid bilayer are contacted with a F-amphiphile. The F-amphiphile inhibits further insertion of the proteins into the lipid bilayer and thereby results in the predetermined number of proteins being present in the lipid bilayer. The predetermined number of membrane proteins may be any number, such as 1, 2, 4, 5, 7, 8, 10, 12, 14, 15, 20, 30, 40, 50, 100 or more proteins. The invention preferably concerns inserting one transmembrane pore or ion channel into the lipid bilayer. In this preferred embodiment, the invention concerns inserting a single transmembrane pore or ion channel into the lipid bilayer.

If the membrane protein is a monomer derived from α-hemolysin (i.e. the sequence shown in SEQ ID NO: 2 or a variant thereof), the predetermined number of membrane proteins is preferably 7. If the membrane protein is a heptameric pore derived from α-hemolysin (i.e. seven subunits having the sequence shown in SEQ ID NO: 2 or a variant thereof), the predetermined number is preferably 1. If the membrane protein is a monomer derived from MspA from Mycobacterium smegmatis (i.e. the sequence shown in SEQ ID NO: 14 or a variant thereof), the predetermined number is preferably 8. If the membrane protein is a octameric pore derived from MspA from Mycobacterium smegmatis (i.e. eight subunits having the sequence shown in SEQ ID NO: 14 or a variant thereof), the predetermined number is preferably 1. If the membrane protein is a monomer derived from Kcv of chlorella virus PBCV-1 (i.e. the sequence shown in SEQ ID NO: 16 or a variant thereof), the predetermined number is preferably 4. If the membrane protein is a tetrameric channel derived from Kcv of chlorella virus PBCV-1 (i.e. four subunits having the sequence shown in SEQ ID NO: 16 or a variant thereof), the predetermined number is preferably 1. These embodiments ensure that one (i.e. a single) pore or channel is present in the lipid bilayer.

It is straightforward to determine how many membrane proteins are present in a lipid bilayers. Methods for doing this are well-known in the art. Single molecule (i.e. monomeric) membrane proteins can be tagged using, for example, fluorescence, radioactivity, tag for antibody, biotin or a spin label. If the one or more proteins form a transmembrane pore, the number of functional pores may be determined using a dye leakage assay. This involves measuring the leakage of dye through pores formed in the lipid bilayer. One implementation of such an assay is described in the Example. Other methods for pores include, but are not limited to, measuring leakage of other substrates, such as radioactive markers, through pores formed in the lipid bilayer, electrical bilayer recordings, or single molecule fluorescence. The numbers of ion channels can be determined using electrical bilayer recordings. Assays for determining the number of G-protein coupled receptors present in a lipid bilayer are also well-known in the art. The number of membrane proteins present in a lipid bilayer can also be predicted as long as the rate of insertion of such proteins into a lipid bilayer is known.

The methods of the invention are advantageous because they allow the number of membrane proteins and hence pores present in a lipid bilayer to be controlled. The number of membrane proteins and pores present in a lipid bilayer can be important if the protein is being studied or if the bilayer forms part of a sensor. Being able to ensure that only one transmembrane pore or ion channel is present in a lipid bilayer allows single-channel studies of the pore to be performed. The presence of only one transmembrane pore or ion channel in a lipid bilayer also allows the production of sensitive sensors, such as those used to sequence nucleic acids. This is discussed in more detail below. It also allows the production of sensors that can detect the presence or absence of a variety of different analytes.

Stochastic sensors can be created by placing a single transmembrane pore or ion channel in a lipid bilayer and measuring voltage-driven ionic transport through the pore or channel in the presence of analyte molecules. The advantages of stochastic sensing include, but are not limited to, the following: (a) a high sensitivity, (b) rapid responses (milliseconds to seconds in the nanomolar concentration range), (c) reversibility, (d) a wide dynamic range, (e) both the concentration and identity of the analyte may be determined, (f) the sensor element need not be highly selective since each analyte produces a characteristic signal, (g) several analytes can be quantitated concurrently by a single sensor, (h) there is a lack of simultaneous competition by similar analytes at the single binding site, (i) fouling does not give a false reading because it generates a signal that is not characteristic of the analyte, (j) there is no loss of signal-to-noise ratio at low analyte concentrations, (h) a digital output facilitates electronic interfacing, (k) the method is self-calibrating and operable without reagents, (l) the signal contains kinetic information.

Other advantages of the invention include:
being able to make lipid bilayers with two or more proteins in defined ratios, for example by providing a bilayer with a known number of proteins A and then adding a known number of proteins B, by the approach described;
being able to build chips with multiple bilayers with optimal Poisson number (37%) of single pores or channels by shutting down incorporation at the optimal time point;
being able to prevent the exchange of proteins between bilayers;
allowing the simple preparation of two populations of lipid vesicle, one with and one without a particular protein in the bilayers; and
being able to stop further permeabilization or limit permeabilization of cells in cell biology experiments or during a cell permeabilization process (e.g. Russo et al., Nature Biotechnology 15, 278-282 (1997); and Eroglu et al., Nature Biotechnology 18, 163-167 (2000)).

### Membrane Protein

The method involves the use of one or more membrane proteins. A membrane protein is a protein that is naturally attached to, or associated with, the membrane of a cell or an organelle. The one or more membrane proteins must be capable of being inserted into the lipid bilayer. Any suitable membrane protein may be used in the method of the invention. The one or more membrane proteins are preferably one or more transmembrane proteins. Suitable transmembrane proteins include, but are not limited to, pores, such as and MspA, ion channels, such as Kcv, and G-protein coupled receptors. The one or more transmembrane proteins may comprise α-helices and/or βstrands.

The method may involve the use of one or more transmembrane pore-forming proteins. A transmembrane pore is a polypeptide or a collection of polypeptides that permits hydrated ions driven by an applied potential to flow from one side of a membrane to the other side of the membrane. In the present invention, the transmembrane pore-forming protein is capable of forming a pore that permits hydrated ions driven by an applied potential to flow from one side of the lipid bilayer to the other. The transmembrane pore preferably permits nucleotides to flow from one side of a membrane, such as a lipid bilayer, to the other along the applied potential. The transmembrane pore preferably allows a nucleic acid, such as DNA or RNA, to be pushed or pulled through the pore.

The transmembrane pore may be a monomer or an oligomer. The pore is preferably made up of several repeating subunits, such as 6, 7 or 8 subunits. The pore is more preferably a tetrameric or heptameric pore. As discussed above, the one or more membrane proteins used in the invention may be an oligomeric pore or one or more pore monomers.

The transmembrane pore typically comprises a barrel or channel through which the ions may flow. The subunits of the pore typically surround a central axis and contribute strands to a transmembrane β barrel or channel or a transmembrane α-helix bundle or channel. The barrel or channel of the pore is preferably greater than 18 angstroms at its widest point. The barrel or channel of the pore is more preferably greater than 20, 25, 30, 35, 40 or 45 angstroms at its widest point. The barrel or channel of the pore is most preferably 46 angstroms at its widest point.

If the transmembrane pore is an oligomer, the one or more membrane proteins used in the methods of the invention may be the oligomer or one or more, such as 2, 3, 4, 5, 6, 7 or 8, monomers. If one or more monomers are used in the method of the invention, it is preferred that sufficient monomers to form one or more pores in the lipid bilayer are used. It is preferred that the monomers self-assemble in the lipid bilayer to form a transmembrane pore through which hydrated ions and preferably nucleotides can flow across the lipid bilayer under an applied potential.

The barrel or channel of the transmembrane pore typically comprises amino acids that facilitate interaction with nucleotides or nucleic acids. These amino acids are preferably located near a constriction of the barrel or channel. The pore typically comprises one or more positively charged amino acids, such as arginine, lysine or histidine. These amino acids typically facilitate the interaction between the pore and nucleotides or nucleic acids. The nucleotide detection can be facilitated with an adaptor. This is discussed in more detail below.

Transmembrane pore-forming proteins for use in accordance with the invention can be derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin A (MspA), outer membrane porin F (OmpF), outer membrane porin G (OmpG), outer membrane phospholipase A and Neisseria autotransporter lipoprotein (NalP). α-helix bundle pores comprise a barrel or channel that is formed from α-helices. Suitable α-helix bundle pores include, but are not limited to, inner membrane proteins and a outer membrane proteins, such as WZA and ClyA toxin.

The one or more transmembrane pore-forming proteins are preferably derived from α-hemolysin (α-HL). The wild type α-HL pore is formed of seven identical monomers or subunits (i.e. it is heptameric). The sequence of one wild type monomer or subunit of α-hemolysin is shown in SEQ ID NO: 2. The one or more transmembrane pore-forming proteins preferably each comprise the sequence shown in SEQ ID NO: 2 or a variant thereof. Amino acids 1, 7 to 21, 31 to 34, 45 to 51, 63 to 66, 72, 92 to 97, 104 to 111, 124 to 136, 149 to 153, 160 to 164, 173 to 206, 210 to 213, 217, 218, 223 to 228, 236 to 242, 262 to 265, 272 to 274, 287 to 290 and 294 of SEQ ID NO: 2 form loop regions. Residues 113 and 147 of SEQ ID NO: 2 form part of a constriction of the barrel or channel of α-HL.

In such embodiments, seven proteins comprising the sequence shown in SEQ ID NO: 2 or a variant thereof are preferably used in the method of the invention. The seven proteins may be the same (homoheptamer) or different (heteroheptamer). The seven proteins typically form a functional pore in the lipid bilayer.

A variant of SEQ ID NO: 2 is a protein that has an amino acid sequence which varies from that of SEQ ID NO: 2 and which retains its pore forming ability. The ability of a variant to form a pore can be assayed using any method known in the art. For instance, the variant may be inserted into a lipid bilayer along with other appropriate subunits and its ability to oligomerise to form a pore may be determined. Methods are known in the art for inserting subunits into membranes, such as lipid bilayers. For example, subunits may be suspended in a purified form in a solution containing a lipid bilayer such that it diffuses to the lipid bilayer and is inserted by binding to the lipid bilayer and assembling into a functional state. Alternatively, subunits may be directly inserted into the membrane using the “pick and place” method described in M. A. Holden, H. Bayley. J. Am. Chem. Soc. 2005, 127, 6502-6503 and International Application No. PCT/GB2006/001057 (published as WO 2006/100484).

The variant may include modifications that facilitate covalent attachment to or interaction with a nucleic acid binding protein. The variant preferably comprises one or more reactive cysteine residues that facilitate attachment to the nucleic acid binding protein. For instance, the variant may include a cysteine at one or more of positions 8, 9, 17, 18, 19, 44, 45, 50, 51, 237, 239 and 287 and/or on the amino or carboxy terminus of SEQ ID NO: 2. Preferred variants comprise a substitution of the residue at position 8, 9, 17, 237, 239 and 287 of SEQ ID NO: 2 with cysteine (K8C, T9C, N17C, K237C, S239C or E287C). The variant is preferably any one of the variants described in International Application No. PCT/GB09/001,690 (published as WO 2010/004273), PCT/GB09/001,679 (published as WO 2010/004265) or PCT/GB10/000,133 (published as WO 2010/086603).

The variant may also include modifications that facilitate any interaction with nucleotides or facilitate orientation of a molecular adaptor as discussed below. The variant may also contain modifications that facilitate covalent attachment of a molecular adaptor.

In particular, the variant preferably has a glutamine at position 139 of SEQ ID NO: 2. The variant preferably has a cysteine at position 119, 121 or 135 of SEQ ID NO: 2. SEQ ID NO: 4 shows the sequence of SEQ ID NO: 2 except that it has an cysteine at position 135 (L135C) and a glutamine at position 139 (N139Q). SEQ ID NO: 4 or a variant thereof may be used to form a pore in accordance with the invention. The variant may have an arginine at position 113 of SEQ ID NO: 2.

The variant may be a naturally occurring variant which is expressed naturally by an organism, for instance by a Staphylococcus bacterium. Alternatively, the variant may be expressed in vitro or recombinantly by a bacterium such as Escherichia coli. Variants also include non-naturally occurring variants produced by recombinant technology. Over the entire length of the amino acid sequence of SEQ ID NO: 2 or 4, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 2 or 4 over the entire sequence. There may be at least 80%, for example at least 85%, 90% or 95%, amino acid identity over a stretch of 200 or more, for example 230, 250, 270 or 280 or more, contiguous amino acids (“hard homology”).

Standard methods in the art may be used to determine homology. For example the UWGCG Package provides the BESTFIT program which can be used to calculate homology, for example used on its default settings (Devereux et al (1984) Nucleic Acids Research 12, p 387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSP's containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Amino acid substitutions may be made to the amino acid sequence of SEQ ID NO: 2 or 4 in addition to those discussed above, for example up to 1, 2, 3, 4, 5, 10, 20 or 30 substitutions. Conservative substitutions may be made. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acid residues of the amino acid sequence of SEQ ID NO: 2 may additionally be deleted from the polypeptides described above. Up to 1, 2, 3, 4, 5, 10, 20 or 30 residues may be deleted, or more.

Variants may fragments of SEQ ID NO: 2 or 4. Such fragments retain pore-forming activity. Fragments may be at least 50, 100, 200 or 250 amino acids in length. A fragment preferably comprises the pore-forming domain of SEQ ID NO: 2 or 4. Fragments typically include residues 119, 121, 135. 113 and 139 of SEQ ID NO: 2 or 4.

One or more amino acids may be alternatively or additionally added to the polypeptides described above. An extension may be provided at the amino terminus or carboxy terminus of the amino acid sequence of SEQ ID NO: 6, 52, 54 or 56 or a variant or fragment thereof. The extension may be quite short, for example from 1 to 10 amino acids in length. Alternatively, the extension may be longer, for example up to 50 or 100 amino acids. A carrier protein may be fused to a subunit or variant.

One or more amino acids may be alternatively or additionally added to the polypeptides described above. An extension may be provided at the amino terminus or carboxy terminus of the amino acid sequence of SEQ ID NO: 2 or 4 or a variant or fragment thereof. The extension may be quite short, for example from 1 to 10 amino acids in length. Alternatively, the extension may be longer, for example up to 50 or 100 amino acids. A carrier protein may be fused to a pore or variant.

As discussed above, a variant of SEQ ID NO: 2 or 4 is a subunit that has an amino acid sequence which varies from that of SEQ ID NO: 2 or 4 and which retains its ability to form a pore. A variant typically contains the regions of SEQ ID NO: 2 or 4 that are responsible for pore formation. The pore forming ability of α-HL, which contains a β-barrel, is provided by β-strands in each subunit. A variant of SEQ ID NO: 2 or 4 typically comprises the regions in SEQ ID NO: 2 that form β-strands. The amino acids of SEQ ID NO: 2 or 4 that form β-strands are discussed above. One or more modifications can be made to the regions of SEQ ID NO: 2 or 4 that form β-strands as long as the resulting variant retains its ability to form a pore. Specific modifications that can be made to the β-strand regions of SEQ ID NO: 2 or 4 are discussed above.

A variant of SEQ ID NO: 2 or 4 preferably includes one or more modifications, such as substitutions, additions or deletions, within its α-helices and/or loop regions. Amino acids that form α-helices and loops are discussed above.

The variant may be modified for example by the addition of histidine or aspartic acid residues to assist its identification or purification or by the addition of a signal sequence to promote their secretion from a cell where the polypeptide does not naturally contain such a sequence.

The one or more transmembrane pore-forming proteins are preferably derived from MspA from Mycobacterium smegmatis. The wild type MspA pore is formed of eight identical monomers or subunits (i.e. it is octameric). The sequence of one wild type monomer or subunit of MspA is shown in SEQ ID NO: 14. The one or more transmembrane pore-forming proteins preferably each comprise the sequence shown in SEQ ID NO: 14 or a variant thereof. In such embodiments, eight proteins comprising the sequence shown in SEQ ID NO: 14 or a variant thereof are preferably used in the method of the invention. The eight proteins may be the same (homooctamer) or different (heterooctamer). The eight proteins typically form a functional pore in the lipid bilayer.

A variant of SEQ ID NO: 14 is a subunit that has an amino acid sequence which varies from that of SEQ ID NO: 14 and which retains its pore forming ability. The ability of a variant to form a pore can be assayed using any method known in the art.

The variant may include modifications that facilitate covalent attachment to or interaction with a nucleic acid binding protein. The variant preferably comprises one or more reactive cysteine residues that facilitate attachment to a nucleic acid binding protein. The variant may also include modifications that facilitate any interaction with nucleotides or facilitate orientation of a molecular adaptor.

The variant may be a naturally occurring variant which is expressed naturally by an organism, for instance by Mycobacterium smegmatis. Alternatively, the variant may be expressed in vitro or recombinantly by a bacterium such as Escherichia coli. Variants also include non-naturally occurring variants produced by recombinant technology. Over the entire length of the amino acid sequence of SEQ ID NO: 14, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 14 over the entire sequence. There may be at least 80%, for example at least 85%, 90% or 95%, amino acid identity over a stretch of 40 or more, for example 50, 60, 70 or 80 or more, contiguous amino acids (“hard homology”). Homology can be measured as described above.

Amino acid substitutions may be made to the amino acid sequence of SEQ ID NO: 14 or 4 in addition to those discussed above, for example up to 1, 2, 3, 4, 5, 10 or 20 substitutions. Conservative substitutions may be made, for example, according to Table 1 above.

One or more amino acid residues of the amino acid sequence of SEQ ID NO: 14 may additionally be deleted from the polypeptides described above. Up to 1, 2, 3, 4, 5, 10, 20 or 30 residues may be deleted, or more.

Variants may fragments of SEQ ID NO: 14. Such fragments retain pore forming activity. Fragments may be at least 50, 60 OR 70 amino acids in length. A fragment preferably comprises the pore forming domain of SEQ ID NO: 14.

One or more amino acids may be alternatively or additionally added to the polypeptides described above. An extension may be provided at the amino terminus or carboxy terminus of the amino acid sequence of SEQ ID NO: 14 or a variant or fragment thereof. The extension may be quite short, for example from 1 to 10 amino acids in length. Alternatively, the extension may be longer, for example up to 20 or 100 40 amino acids. A carrier protein may be fused to a pore-forming protein or variant.

As discussed above, a variant of SEQ ID NO: 14 is a subunit that has an amino acid sequence which varies from that of SEQ ID NO: 14 and which retains its ability to form a pore. A variant typically contains the regions of SEQ ID NO: 14 that are responsible for pore formation. The pore forming ability of MspA, which contains a β-barrel, is provided by β-strands in each subunit. A variant of SEQ ID NO: 14 typically comprises the regions in SEQ ID NO: 14 that form β-strands. One or more modifications can be made to the regions of SEQ ID NO: 14 that form β-strands as long as the resulting variant retains its ability to form a pore. A variant of SEQ ID NO: 14 preferably includes one or more modifications, such as substitutions, additions or deletions, within its non-β-strands regions.

In another embodiment, the method involves the use of one or more ion channel proteins. An ion channel is a polypeptide or a collection of polypeptides that permits non-hydrated ions driven by an applied potential to flow from one side of a membrane to the other side of the membrane. In the present invention, the one or more ion channel proteins are capable of forming a channel that permits non-hydrated ions driven by an applied potential to flow from one side of the lipid bilayer to the other.

The ion channel may be a monomer or an oligomer. The channel is preferably made up of several repeating subunits, such as 4, 5 or 6 subunits. The channel is more preferably a tetrameric pore. As discussed above, the one or more membrane proteins used in the invention may be an oligomeric channel or one or more channel monomers. The ion channel typically comprises a transmembrane α-helix bundle.

If the ion channel is an oligomer, the one or more membrane proteins used in the methods of the invention may be the oligomer or one or more, such as 2, 3, 4, 5, 6, 7 or 8, monomers. If one or more monomers are used in the method of the invention, it is preferred that sufficient monomers to form one or more ion channels in the lipid bilayer are used. It is preferred that the monomers self-assemble in the lipid bilayer to form a transmembrane channel through which non-hydrated ions can flow across the lipid bilayer under an applied potential.

The one or more membrane proteins may be derived from any ion channel. Suitable channels include, but are not limited to, sodium channels, potassium channels, calcium channels and magnesium channels. The channel may be voltage-gated, ligand-gated or gated by another mechanism, such as calcium or other ions, light or mechanical stimulation. The one or more membrane proteins are preferably not derived from mechanosensitive channel of large conductance, MscL, of Escherichia coli.

The one or more ion channel proteins are preferably derived from Kcv of chlorella virus PBCV-1. The wild type Kcv channel is formed of four identical monomers or subunits (i.e. it is tetrameric). The sequence of one wild type monomer or subunit of Kcv is shown in SEQ ID NO: 16. The one or more ion channel proteins preferably each comprise the sequence shown in SEQ ID NO: 16 or a variant thereof. In such embodiments, four proteins each comprising the sequence shown in SEQ ID NO: 16 or a variant thereof are preferably used in the method of the invention. The four proteins may be the same (homotetramer) or different (heterotetramer). The four proteins typically form a functional channel in the lipid bilayer.

A variant of SEQ ID NO: 16 is a subunit that has an amino acid sequence which varies from that of SEQ ID NO: 16 and which retains its channel forming ability. The ability of a variant to form a channel can be assayed using any method known in the art. For instance, the variant may be inserted into a membrane along with other appropriate subunits and its ability to oligomerise to form a channel may be determined. Methods are known in the art for inserting subunits into membranes, such as lipid bilayers. These are discussed above.

The variant may be a naturally occurring variant which is expressed naturally by an organism, for instance by chlorella virus PBCV-1. Alternatively, the variant may be expressed in vitro or recombinantly by a bacterium such as Escherichia coli. Variants also include non-naturally occurring variants produced by recombinant technology. Over the entire length of the amino acid sequence of SEQ ID NO: 16, a variant will preferably be at least 50% homologous to that sequence based on amino acid identity. More preferably, the variant polypeptide may be at least 55%, at least 60%, at least 65%, at least 70%, at least 75%, at least 80%, at least 85%, at least 90% and more preferably at least 95%, 97% or 99% homologous based on amino acid identity to the amino acid sequence of SEQ ID NO: 16 over the entire sequence. There may be at least 80%, for example at least 85%, 90% or 95%, amino acid identity over a stretch of 40 or more, for example 50, 60, 70 or 80 or more, contiguous amino acids (“hard homology”). Homology can be measured as described above.

Amino acid substitutions may be made to the amino acid sequence of SEQ ID NO: 16 or 4 in addition to those discussed above, for example up to 1, 2, 3, 4, 5, 10 or 20 substitutions. Conservative substitutions may be made, for example, according to Table 1 above.

One or more amino acid residues of the amino acid sequence of SEQ ID NO: 16 may additionally be deleted from the polypeptides described above. Up to 1, 2, 3, 4, 5, 10, 20 or 30 residues may be deleted, or more.

Variants may fragments of SEQ ID NO: 16. Such fragments retain channel forming activity. Fragments may be at least 50, 60 OR 70 amino acids in length. A fragment preferably comprises the channel forming domain of SEQ ID NO: 16.

One or more amino acids may be alternatively or additionally added to the polypeptides described above. An extension may be provided at the amino terminus or carboxy terminus of the amino acid sequence of SEQ ID NO: 16 or a variant or fragment thereof. The extension may be quite short, for example from 1 to 10 amino acids in length. Alternatively, the extension may be longer, for example up to 20 or 100 40 amino acids. A carrier protein may be fused to a channel-forming protein or variant.

As discussed above, a variant of SEQ ID NO: 16 is a subunit that has an amino acid sequence which varies from that of SEQ ID NO: 16 and which retains its ability to form a channel. A variant typically contains the regions of SEQ ID NO: 16 that are responsible for channel formation. The channel forming ability of Kcv, which contains an α-helix bundle, is provided by α-helixes in each subunit. A variant of SEQ ID NO: 16 typically comprises the regions in SEQ ID NO: 16 that form α-helixes. One or more modifications can be made to the regions of SEQ ID NO: 16 that form α-helixes as long as the resulting variant retains its ability to form a channel. Specific modifications that can be made to the α-helix regions of SEQ ID NO: 16 are discussed above.

A variant of SEQ ID NO: 16 preferably includes one or more modifications, such as substitutions, additions or deletions, within its non-α-helix regions.

Any of the variants discussed above may be modified for example by the addition of histidine or aspartic acid residues to assist its identification or purification or by the addition of a signal sequence to promote their secretion from a cell where the polypeptide does not naturally contain such a sequence.

The membrane protein may be labelled with a revealing label. The revealing label may be any suitable label which allows the pore to be detected. Suitable labels include, but are not limited to, fluorescent molecules, radioisotopes, e.g. ¹²⁵I, ³⁵S, ¹⁴C, enzymes, antibodies, antigens, polynucleotides and ligands such as biotin.

The one or more membrane proteins may be isolated from a pore producing organism, such as Staphylococcus aureus or chlorella virus PBCV-1, or made synthetically or by recombinant means. For example, membrane proteins may be synthesised by in vitro translation and transcription. The amino acid sequence of the proteins may be modified to include non-naturally occurring amino acids or to increase the stability of the proteins. When the proteins are produced by synthetic means, such amino acids may be introduced during production. The proteins may also be altered following either synthetic or recombinant production. Native chemical ligations, which can combine expression and synthesis, may also be used (e.g. Bayley et al., ACS Chem. Biol. 4, 983-985 (2009)).

The membrane proteins may also be produced using D-amino acids. For instance, the membrane proteins may comprise a mixture of L-amino acids and D-amino acids. This is conventional in the art for producing such proteins or peptides.

The one or more membrane proteins may also contain other non-specific chemical modifications as long as they do not interfere with their ability to form a pore. A number of non-specific side chain modifications are known in the art and may be made to the side chains of the pores. Such modifications include, for example, reductive alkylation of amino acids by reaction with an aldehyde followed by reduction with NaBH₄, amidination with methylacetimidate or acylation with acetic anhydride. The modifications to the membrane proteins can be made after its expression or after it has been inserted into a lipid bilayer.

The one or more membrane proteins can be produced using standard methods known in the art. Polynucleotide sequences encoding a membrane protein may be isolated and replicated using standard methods in the art.

Polynucleotide sequences may be isolated and replicated using standard methods in the art. Chromosomal DNA may be extracted from a pore producing organism, such as Staphylococcus aureus or chlorella virus PBCV-1. The gene encoding the protein may be amplified using PCR involving specific primers. The amplified sequences may then be incorporated into a recombinant replicable vector such as a cloning vector. The vector may be used to replicate the polynucleotide in a compatible host cell. Thus polynucleotide sequences encoding the membrane protein may be made by introducing a polynucleotide encoding the protein into a replicable vector, introducing the vector into a compatible host cell, and growing the host cell under conditions which bring about replication of the vector. The vector may be recovered from the host cell. Suitable host cells for cloning of polynucleotides are known in the art and described in more detail below.

The polynucleotide sequence may be cloned into suitable expression vector. In an expression vector, the polynucleotide sequence encoding a protein is typically operably linked to a control sequence which is capable of providing for the expression of the coding sequence by the host cell. Such expression vectors can be used to express a protein.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The expression vector may then be introduced into a suitable host cell. Thus, a protein can be produced by inserting a polynucleotide sequence encoding a protein into an expression vector, introducing the vector into a compatible bacterial host cell, and growing the host cell under conditions which bring about expression of the polynucleotide sequence. The recombinantly-expressed pore may self-assemble into a pore in the host cell membrane. Alternatively, the recombinant pore produced in this manner may be isolated from the host cell and inserted into another membrane. When producing an oligomeric pore comprising different monomers, the different monomers may be expressed separately in different host cells as described above, removed from the host cells and assembled into a pore in a separate membrane, such as a rabbit cell membrane.

The vectors may be for example, plasmid, virus or phage vectors provided with an origin of replication, optionally a promoter for the expression of the said polynucleotide sequence and optionally a regulator of the promoter. The vectors may contain one or more selectable marker genes, for example an ampicillin resistance gene. Promoters and other expression regulation signals may be selected to be compatible with the host cell for which the expression vector is designed. A T7, trc, lac, ara or λ_{L} promoter is typically used.

The host cell typically expresses the protein at a high level. Host cells transformed with a polynucleotide sequence will be chosen to be compatible with the expression vector used to transform the cell. The host cell is typically bacterial and preferably E. coli. Any cell with a λ DE3 lysogen, for example C41 (DE3), BL21 (DE3), JM109 (DE3), B834 (DE3), TUNER, Origami and Origami B, can express a vector comprising the T7 promoter.

A membrane protein may be produced in large scale following purification by any protein liquid chromatography system from pore producing organisms or after recombinant expression as described below. Typical protein liquid chromatography systems include FPLC, AKTA systems, the Bio-Cad system, the Bio-Rad BioLogic system and the Gilson HPLC system.

In a further embodiment, at least one of the membrane proteins is attached to a nucleic acid binding protein. This is one way of allowing a lipid bilayer having the protein inserted therein to be used to sequence nucleic acids. Examples of nucleic acid binding proteins include, but are not limited to, nucleic acid handling enzymes, such as nucleases, polymerases, topoisomerases, ligases and helicases, and non-catalytic binding proteins such as those classified by SCOP (Structural Classification of Proteins) under the Nucleic acid-binding protein superfamily (50249). The nucleic acid binding protein is preferably modified to remove and/or replace cysteine residues as described in International Application No. PCT/GB10/000,133 (published as WO 2010/086603).

A nucleic acid is a macromolecule comprising two or more nucleotides. The nucleic acid bound by the protein may comprise any combination of any nucleotides. The nucleotides can be naturally occurring or artificial. The nucleotide can be oxidized or methylated. A nucleotide typically contains a nucleobase, a sugar and at least one phosphate group. The nucleobase is typically heterocyclic. Nucleobases include, but are not limited to, purines and pyrimidines and more specifically adenine, guanine, thymine, uracil and cytosine. The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The nucleotides are preferably selected from AMP, TMP, GMP, CMP, UMP, dAMP, dTMP, dGMP or dCMP.

The nucleic acid can be deoxyribonucleic acid (DNA) or ribonucleic acid (RNA). The nucleic acid may be any synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains. The nucleic acid bound by the protein is preferably single stranded, such as cDNA, RNA, GNA, TNA or LNA. The nucleic acid bound by the protein is preferably double stranded, such as DNA. Proteins that bind single stranded nucleic acids may be used to sequence double stranded DNA as long as the double stranded DNA is dissociated into a single strand before it is bound by the protein.

Preferred nucleic acid binding proteins for use in the invention include exonuclease I from E. coli (SEQ ID NO: 6), exonuclease III enzyme from E. coli (SEQ ID NO: 8), RecJ from T. thermophilus (SEQ ID NO: 10) and bacteriophage lambda exonuclease (SEQ ID NO: 12) and variants thereof. Three identical subunits of SEQ ID NO: 12 interact to form a trimer exonuclease. The enzyme is most preferably based on exonuclease I from E. coli (SEQ ID NO: 6). The variant is preferably modified to facilitate attachment to the membrane protein and may be any of those discussed in International Application No. PCT/GB09/001,679 (published as WO 2010/004265) or PCT/GB10/000,133 (published as WO 2010/086603). The protein may be any of SEQ ID NOs: 8, 10, 12, 14, 16, 18, 20, 22, 24, 26, 28, 30, 32, 34, 36, 38, 40, 42, 44, 46, 48 and 50 described in International Application No. PCT/GB10/000,133 (published as WO 2010/086603) or a variant thereof discussed in that International application. The nucleic acid binding protein may be attached to the membrane protein in any manner and is preferably attached as described in International Application No. PCT/GB09/001,679 (published as WO 2010/004265) or PCT/GB10/000,133 (published as WO 2010/086603).

### Lipid Bilayer

The one or more membrane proteins are contacted with a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. In particular, lipid bilayers can be used to detect the presence of membrane pores or channels or can be used in stochastic sensing in which the response of a membrane protein to a molecule or physical stimulus is used to perform sensing of that molecule or stimulus. The lipid bilayer may be any lipid bilayer. Suitable lipid bilayers include, but are not limited to, a planar lipid bilayer, a supported bilayer or a liposome.

A planar lipid bilayer is typically formed across an aperture in a membrane. The membrane can be made from any material including, but not limited to, a polymer, glass and a metal. The membrane is preferably made from a material that forms a barrier to the flow of ions. Suitable materials include, but are not limited to, polycarbonate (PC), polytetrafluoroethylene (PTFE), polyethylene, polypropylene, nylon and polyethylene naphthalate (PEN), polyvinylchloride (PVC), polyacrylonitrile (PAN), polyether sulphone (PES), polyimide, polystyrene, polyvinylfluoride (PVF), polyethylene telephthalate (PET), aluminized PET, nitrocellulose, polyetheretherketone (PEEK) and fluoroethylkene polymer (FEP). The membrane is preferably made from polycarbonate or PTFE.

The membrane is sufficiently thin to facilitate formation of the lipid bilayer across an aperture as described below. Typically the thickness will be in the range of 10 nm to 1 mm. The membrane is preferably 0.1 μm to 25 μm thick.

The membrane is preferably pre-treated to make the lipids and the aperture more compatible such that the lipid bilayer forms more easily that it would in the absence of pre-treatment. The membrane is preferably pre-treated to increase its affinity to lipids and thereby allow the lipid bilayer to form more easily.

Any treatment that modifies the surface of the membrane surrounding the aperture to increase its affinity to lipids may be used. The membrane is typically pre-treated with long chain organic molecules in an organic solvent. Suitable long chain organic molecules include, but are not limited to, n-decane, hexadecane, hexadecane mixed with one or more of the lipids discussed below, iso-eicosane, octadecane, squalene, fluoroinated oils (suitable for use with fluorinated lipids), alkyl-silane (suitable for use with a glass membrane) and alkyl-thiols (suitable for use with a metallic membrane). Suitable solvents include, but are not limited to, pentane, hexane, heptane, octane, decane, iso-ecoisane and toluene. The membrane is typically pre-treated with from 0.1% (v/v) to 50% (v/v), such as 0.3%, 1% or 3% (v/v), hexadecane in pentane. The volume of hexadecane in pentane used is typically from 0.1 μl to 10 μl. The hexadecane can be mixed with one or more lipids. For instance, the hexadecane can be mixed with any of the lipids discussed below. The hexadecane is preferably mixed with diphantytanoyl-sn-glycero-3-phosphocoline (DPhPC). Preferably, the aperture is treated with 2 μl of 1% (v/v) hexadecane and 0.6 mg/ml lipid, such as DPhPC, in pentane.

Some specific pretreatments are set out in Table 3 by way of example and without limitation.

TABLE 3

| | Volumes applied by capillary |
|---|---|
| Pretreatment formulation | pipette |
| 0.3% hexadecane in pentane | 2x 1 μl |
| 1% hexadecane in pentane | 2x 0.5 μl; 2x 0.5 μl; 1 μl; 2x 1 μl; 2x |
| | 1 μl; 2 μl; 2x 2 μl; 5 μl |
| 3% hexadecane in pentane | 2x 1 μl; 2 μl |
| 10% hexadecane in pentane | 2x 1 μl; 2 μl; 5 μl |
| 0.5% hexadecane + 5 mg/ml | 5 μl |
| DPhPC lipid in pentane | |
| 1.0% hexadecane + 0.6 mg/ml | 2x 0.5 μl |
| DPhPC lipid in pentane | |
| 1.5% hexadecane + 5 mg/ml | 2 μl; 2x 1 μl |
| DPhPC lipid in pentane | |

The precise volume of pretreatment substance required depends on the pretreatment both the size of the aperture, the formulation of the pretreatment, and the amount and distribution of the pretreatment when it dries around the aperture. In general increasing the amount of pretreatment (i.e. by volume and/or by concentration) improves the effectiveness, but too much pretreatment can block the aperture. As the diameter of the aperture is decreased, the amount of pretreatment required also decreases. The distribution of the pretreatment can also affect effectiveness, this being dependent on the method of deposition, and the compatibility of the membrane surface chemistry.

The relationship between the pretreatment and the ease and stability of bilayer formation is therefore complex, depending on a complex cyclic interaction between the aperture dimensions, the membrane surface chemistry, the pretreatment formulation and volume, and the method of deposition. The temperature dependent stability of the pretreated aperture further complicates this relationship. However, the pretreatment may be optimised by routine trial and error to enable bilayer formation immediately upon first exposure of the dry aperture to the lipid monolayer at the liquid interface.

If the membrane is made from a material that forms a barrier to the flow of ions, the aperture allows the movement of ions between from the chamber. The aperture may be any size and shape which is capable of supporting a lipid bilayer. The aperture preferably has a diameter in at least one dimension which is 25 μm or less. This preferred size of aperture results in the formation of a lipid bilayer with increased stability. This means that the method of the invention can form stable lipid bilayers and that the device can be used in situations where the lipid bilayer is likely to encounter mechanical or other forces. For instance, it can be used as a hand-held device. The preferred size of aperture also allows the lipid bilayer to form more easily. In particular, it allows the formation of a lipid bilayer across the aperture following a single pass of the lipid/solution interface and removes the need to move the lipid/solution interface back and forth past the aperture.

The aperture may be created using any method. Suitable methods include, but are not limited to, spark generation and laser drilling.

Preferred combinations of membrane and aperture for use in accordance with the invention are shown in the Table 4 which sets out in the first column the thickness and material of the membrane and in the second column the diameter and method of forming the aperture.

TABLE 4

| Septum | Aperture |
|---|---|
| 6 μm thick biaxial | 25 μm diameter spark-generated hole |
| polycarbonate | |
| 6 μm thick biaxial | 20 μm diameter laser-drilled tapered hole |
| polycarbonate | |
| 6 μm thick biaxial | 10 μm diameter laser-drilled tapered hole |
| polycarbonate | |
| 5 μm thick PTFE | 10 μm diameter spark-generated holes |
| 5 μm thick PTFE | 10 μm diameter laser-drilled tapered hole |
| 5 μm thick PTFE | 5 μm diameter laser-drilled tapered hole |
| 10 μm thick HD polyethylene | 15 μm diameter spark-generated hole |
| 4 μm thick Polypropylene | 15 μm diameter spark-generated hole |
| 25 μm thick Nylon (6,6) | 20 μm diameter spark-generated hole |
| 1.3 μm thick PEN | 30 μm diameter spark-generated hole |
| 14 μm thick conductive | 30 μm diameter spark-generated hole |
| polycarbonate | |
| 7 μm thick PVC | 20 μm diameter laser-drilled hole |

Methods for forming lipid bilayers are known in the art. Suitable methods are disclosed in the Example. Lipid bilayers are commonly formed by the method of Montal and Mueller (Proc. Natl. Acad. Sci. USA., 1972; 69: 3561-3566), in which a lipid monolayer is carried on aqueous solution/air interface past either side of an aperture which is perpendicular to that interface. The lipid is normally added to the surface of an aqueous electrolyte solution by first dissolving it in an organic solvent and then allowing a drop of the solvent to evaporate on the surface of the aqueous solution on either side of the aperture. Once the organic solvent has evaporated, the solution/air interfaces on either side of the aperture are physically moved up and down past the aperture until a bilayer is formed.

The method of Montal & Mueller is popular because it is a cost-effective and relatively straightforward method of forming good quality lipid bilayers that are suitable for protein pore insertion. Other common methods of bilayer formation include tip-dipping, painting bilayers and patch-clamping of liposome bilayers.

Tip-dipping bilayer formation entails touching the aperture surface (for example, a pipette tip) onto the surface of a test solution that is carrying a monolayer of lipid. Again, the lipid monolayer is first generated at the solution/air interface by allowing a drop of lipid dissolved in organic solvent to evaporate at the solution surface. The bilayer is then formed by the Langmuir-Schaefer process and requires mechanical automation to move the aperture relative to the solution surface.

For painted bilayers, a drop of lipid dissolved in organic solvent is applied directly to the aperture, which is submerged in an aqueous test solution. The lipid solution is spread thinly over the aperture using a paintbrush or an equivalent. Thinning of the solvent results in formation of a lipid bilayer. However, complete removal of the solvent from the bilayer is difficult and consequently the bilayer formed by this method is less stable and more prone to noise during electrochemical measurement.

Patch-clamping is commonly used in the study of biological cell membranes. The cell membrane is clamped to the end of a pipette by suction and a patch of the membrane becomes attached over the aperture. The method has been adapted for producing lipid bilayers by clamping liposomes which then burst to leave a lipid bilayer sealing over the aperture of the pipette. The method requires stable, giant and unilamellar liposomes and the fabrication of small apertures in materials having a glass surface.

Liposomes can be formed by sonication, extrusion or the Mozafari method (Colas et al. (2007) Micron 38:841-847).

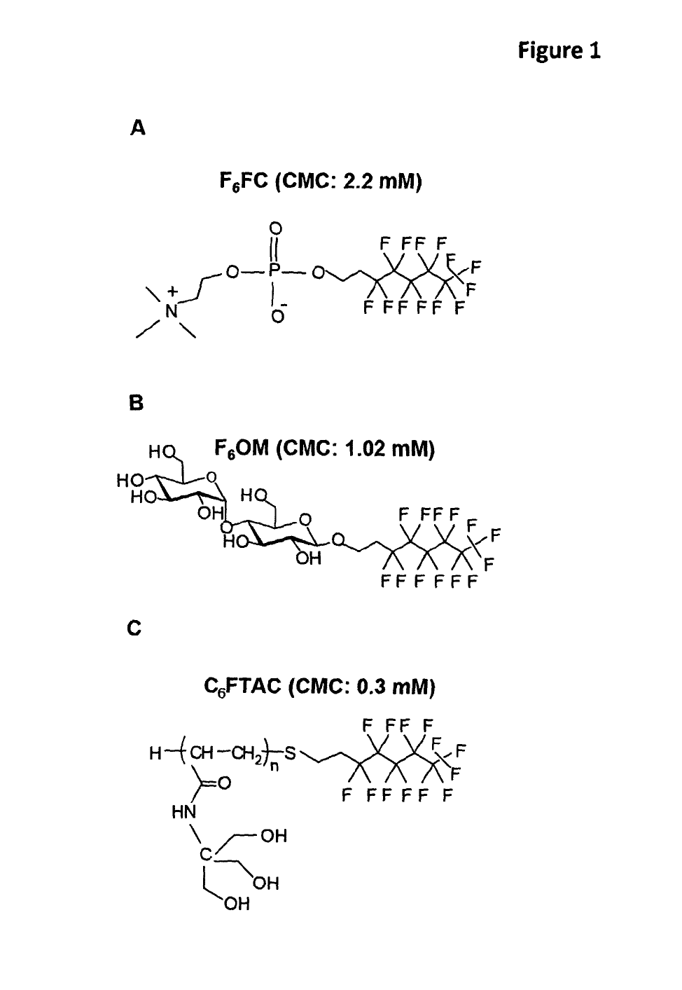

In a preferred embodiment, the lipid bilayer is formed as described in International Application No. PCT/GB08/000,563 (published as WO 2008/102121). Advantageously in this method, the lipid bilayer is formed from dried lipids. Even when dried to a solid state, the lipids will typically contain trace amounts of residual solvent. Dried lipids are preferably lipids that comprise less than 50 wt % solvent, such as less than 40 wt %, less than 30 wt %, less than 20 wt %, less than 15 wt %, less than 10 wt % or less than 5 wt % solvent. In a most preferred embodiment, the lipid bilayer is formed across an aperture in a cell device as shown in FIG. 1 of International Application No. PCT/GB08/000,563 (published as WO 2008/102121).

A lipid bilayer is formed from two opposing layers of lipids. The two layers of lipids are arranged such that their hydrophobic tail groups face towards each other to form a hydrophobic interior. The hydrophilic head groups of the lipids face outwards towards the aqueous environment on each side of the bilayer. The bilayer may be present in a number of lipid phases including, but not limited to, the liquid disordered phase (fluid lamellar), liquid ordered phase, solid ordered phase (lamellar gel phase, interdigitated gel phase) and planar bilayer crystals (lamellar sub-gel phase, lamellar crystalline phase).

Any lipids that form a lipid bilayer may be used. The lipids are chosen such that a lipid bilayer having the required properties, such surface charge, ability to support membrane proteins, packing density or mechanical properties, is formed. The lipids can comprise one or more different lipids. For instance, the lipids can contain up to 100 lipids. The lipids preferably contain 1 to 10 lipids. The lipids may comprise naturally-occurring lipids and/or artificial lipids.

The lipids typically comprise a head group, an interfacial moiety and two hydrophobic tail groups which may be the same or different. Suitable head groups include, but are not limited to, neutral head groups, such as diacylglycerides (DG) and ceramides (CM); zwitterionic head groups, such as phosphatidylcholine (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-Propane (TAP). Suitable interfacial moieties include, but are not limited to, naturally-occurring interfacial moieties, such as glycerol-based or ceramide-based moieties. Suitable hydrophobic tail groups include, but are not limited to, saturated hydrocarbon chains, such as lauric acid (n-Dodecanolic acid), myristic acid (n-Tetradecononic acid), palmitic acid (n-Hexadecanoic acid), stearic acid (n-Octadecanoic) and arachidic (n-Eicosanoic); unsaturated hydrocarbon chains, such as oleic acid (cis-9-Octadecanoic); and branched hydrocarbon chains, such as phytanoyl. The length of the chain and the position and number of the double bonds in the unsaturated hydrocarbon chains can vary. The length of the chains and the position and number of the branches, such as methyl groups, in the branched hydrocarbon chains can vary. The hydrophobic tail groups can be linked to the interfacial moiety as an ether or an ester.

The lipids can also be chemically-modified. The head group or the tail group of the lipids may be chemically-modified. Suitable lipids whose head groups have been chemically-modified include, but are not limited to, PEG-modified lipids, such as 1,2-Diacyl-sn-Glycero-3-Phosphoethanolamine-N-[Methoxy(Polyethylene glycol)-2000]; functionalised PEG Lipids, such as 1,2-Distearoyl-sn-Glycero-3 Phosphoethanolamine-N-[Biotinyl(Polyethylene Glycol) 2000]; and lipids modified for conjugation, such as 1,2-Dioleoyl-sn-Glycero-3-Phosphoethanolamine-N-(succinyl) and 1,2-Dipalmitoyl-sn-Glycero-3-Phosphoethanolamine-N-(Biotinyl). Suitable lipids whose tail groups have been chemically-modified include, but are not limited to, polymerisable lipids, such as 1,2-bis(10,12-tricosadiynoyl)-sn-Glycero-3-Phosphocholine; fluorinated lipids, such as 1-Palmitoyl-2-(16-Fluoropalmitoyl)-sn-Glycero-3-Phosphocholine; deuterated lipids, such as 1,2-Dipalmitoyl-D62-sn-Glycero-3-Phosphocholine; and ether linked lipids, such as 1,2-Di-O-phytanyl-sn-Glycero-3-Phosphocholine.

The lipids typically comprise one or more additives that will affect the properties of the lipid bilayer. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-Acyl-2-Hydroxy-sn-Glycero-3-Phosphocholine; and ceramides. The lipid preferably comprises cholesterol and/or ergosterol when membrane proteins are to be inserted into the lipid bilayer.

The lipid-to-protein ratio used in the method of the invention is preferably lower than 40:1 (w/w), such as lower than 30:1 (w/w), lower than 20:1 (w/w), lower than 10:1 (w/w) or lower than 5:1 (w/w). The lipid-to-protein ratio used in the method of the invention is most preferably 1:1 (w/w).

### Fluoroinated Amphiphiles

The one or more membrane proteins and the lipid bilayer are contacted with a fluorinated amphiphile (F-amphiphile). A F-amphiphile comprises (a) a polar head group and (b) a hydrophobic tail comprising a fluorinated chain. The polar head group may be any polar head. Suitable polar head groups include, but are not limited to, zwitterionic head groups, positive or negative ionic head groups or non-ionic head groups, such as non-ionic disaccharide head groups and non-ionic polymeric head groups. The hydrophobic tail typically comprises an alkyl chain of at least 6 carbon atoms in length, such as 8, 10 12, 14, 16 or 20 or more carbon atoms in length. The alkyl chain may be linear or branched. The alkyl chain is fluorinated. It may comprise at least 10, such as at least 12, at least 15, at least 20, at least 25 or at least 30, fluorine atoms. The number of fluorine atoms will typically be odd because the CF₃— group terminates.

The F-amphiphile is preferably F-fos-choline (F₆FC) with a zwitterionic head group, F-octyl maltoside (F₆OM) with a non-ionic disaccharide head group and C₆F₁₃C₂H₄—S-poly[tris(hydroxymethyl)aminomethane] (F₆TAC) with a non-ionic polymeric head group. These are shown in FIG. 1.

The lipid bilayer typically separates two compartments, the cis and trans compartments. The F-amphiphile is preferably contacted with the proteins and the lipid bilayer via the cis side. The F-amphiphile is typically added in an aqueous solution. Suitable aqueous solutions are discussed below in the sections concerning conditions. In such embodiments, an equal volume of water is typically added to the trans compartment.

The F-amphiphile is typically contacted with the proteins and lipid bilayer at a concentration greater than the critical micelle concentration (CMC). The F-amphiphile is preferably contacted with the protein and lipid bilayer at a concentration at least five times greater than the CMC, such as 10, 15 or 20 times or more than the CMC. The CMC is determined by measuring surface tension, by measuring the conductivity of the solution, using dye spectral shifts or using literature values. These are routine methods in the art.

### Conditions

In the first embodiment of the invention, the proteins and lipid bilayer are contacted with a fluorinated amphiphile (F-amphiphile) under conditions that in the absence of the F-amphiphile allow the insertion of the protein into the lipid bilayer. In the second embodiment of the invention, more than the pre-determined number of proteins are contacted with the lipid bilayer under conditions that allow the insertion of the proteins into the lipid bilayer. Such conditions are known in the art.

The membrane proteins typically spontaneously insert into the lipid bilayer in the presence of an aqueous solution. This avoids the need to actively insert the protein into the lipid bilayer by physically carrying the protein to the bilayer. In another embodiment, the membrane protein is deposited on an internal surface of a chamber as described in International Application No. PCT/GB08/000,563 (published as WO 2008/102121). The aqueous solution collects the membrane proteins from the surface and allows them to insert into the lipid bilayer. In such an embodiment, the membrane proteins are preferably dried. Even when dried to a solid state, the protein will typically contain trace amounts of residual solvent. Dried membrane proteins are preferably proteins that comprise less than 20 wt % solvent, such as less than 15 wt %, less than 10 wt % or less than 5 wt % solvent.

Any aqueous solution that allows the membrane proteins to insert into the lipid bilayer may be used. The aqueous solution is typically a physiologically acceptable solution. The physiologically acceptable solution is typically buffered to a pH of 3 to 9. The pH of the solution will be dependent on the lipids used and the final application of the lipid bilayer. Suitable buffers include, but are not limited, to phosphate buffered saline (PBS), N-2-Hydroxyethylpiperazine-N′-2-Ethanesulfonic Acid (HEPES) buffered saline, piperazine-1,4-Bis-2-Ethanesulfonic Acid (PIPES) buffered saline, 3-(n-Morpholino)Propanesulfonic Acid (MOPS) buffered saline and Tris(Hydroxymethyl)aminomethane (TRIS) buffered saline. By way of example, in one implementation, the aqueous solution may be 10 mM PBS containing 1.0M sodium chloride (NaCl) and having a pH of 6.9. By way of another example, in one implementation, the aqueous solution may be 200 mM KCl, 10 mM HEPES, pH 7.0. By way of a further example, in one implementation, the aqueous solution may be 300 mM NaCl, 2.7 mM KCl, 10 mM Na2HPO4, 2 mM KH2PO4, pH 7.4.

### Lipid Bilayers

The invention also provides a lipid bilayer having a predetermined number of membrane proteins inserted therein. The lipid bilayer preferably has a single membrane protein, transmembrane pore or ion channel inserted therein. The lipid bilayer may be used for a variety of purposes. The lipid bilayer may be used for in vitro investigation of membrane proteins by single-channel recording. The lipid bilayer may be used as a biosensor to detect the presence of a range of substances. The lipid bilayer may be used to detect the presence or absence of membrane pores or channels in a sample. The presence of the pore or channel may be detected as a change in the current flow across the lipid bilayer as the pore or channel inserts into the lipid bilayer. The lipid bilayer preferably contains membrane protein and is used to detect the presence or absence of a molecule or stimulus using stochastic sensing. The lipid bilayer may be used for a range of other purposes, such as studying the properties of molecules known to be present (e.g. DNA sequencing or drug screening), or separating components for a reaction.

The invention also provides a sensing device comprising one or more lipid bilayers of the invention. The sensing device may comprise other lipid bilayers in addition to the one of more lipid bilayers of the invention. Preferred sensing devices include chips and any of the devices described below.

### Stochastic Sensing

A lipid bilayer of the invention may be used to determine the presence of absence of an analyte. The lipid bilayer comprises at least one transmembrane pore or ion channel. Any number of pores or channels may be present in the lipid bilayer as discussed above. The lipid bilayer preferably comprises only one transmembrane pore or only one ion channel. Stochastic sensing using pores is well-known in the art. The use of ion channels in molecular sensing has also been demonstrated (Moreau et al. (2008). Nat Nanotechnol 3, 620-625).

The method comprises contacting the analyte with a lipid bilayer of the invention, which comprises a pore or channel, so that the analyte interacts with the pore or channel and measuring the current passing through the pore or channel during the interaction and thereby determining the presence or absence of the analyte. Any of the transmembrane pores or ion channels discussed above can be used. The benefits associated with using a transmembrane pore or channel to detect an analyte is discussed above.

The analyte is present if the current flows through the pore or channel in a manner specific for the analyte (i.e. if a distinctive current associated with the analyte is detected flowing through the pore or channel). The analyte is absent if the current does not flow through the pore or channel in a manner specific for the analyte.

The invention therefore involves stochastic sensing of an analyte. The invention can be used to differentiate analytes of similar structure on the basis of the different effects they have on the current passing through the pore or channel. The invention can also be used to measure the concentration of a particular analyte in a sample.

The invention may also be used in a sensor that uses many or thousands of pores or channels in bulk sensing applications.

The method may be carried out using any suitable lipid bilayer system in which a pore or channel is inserted into a lipid bilayer. The method is typically carried out using (i) an artificial bilayer comprising a pore or channel, (ii) an isolated, naturally-occurring lipid bilayer comprising a pore or channel, or (iii) a cell having a pore or channel inserted therein. The method is preferably carried out using an artificial lipid bilayer. The bilayer may comprise other transmembrane and/or intramembrane proteins as well as other molecules in addition to the pore or channel. Suitable apparatus and conditions are discussed below with reference to the sequencing embodiments of the invention. The method of the invention is typically carried out in vitro.

During the interaction between the analyte and the pore, the analyte affects the current flowing through the pore or channel in a manner specific for that analyte. For example, a particular analyte will reduce the current flowing through the pore or channel for a particular mean time period and to a particular extent. In other words, the current flowing through the pore or channel is distinctive for a particular analyte. Control experiments may be carried out to determine the effect a particular analyte has on the current flowing through the pore or channel. Results from carrying out the method of the invention on a test sample can then be compared with those derived from such a control experiment in order to identify a particular analyte in the sample or determine whether a particular analyte is present in the sample. The frequency at which the current flowing through the pore or channel is affected in a manner indicative of a particular analyte can be used to determine the concentration of that analyte in the sample.

The analyte can be any substance in a sample. Suitable analytes include, but are not limited to, metal ions, inorganic salts, polymers, such as a polymeric acids or bases, dyes, bleaches, pharmaceuticals, diagnostic agents, recreational drugs, explosives and environmental pollutants.

The analyte can be an analyte that is secreted from cells. Alternatively, the analyte can be an analyte that is present inside cells such that the analyte must be extracted from the cells before the invention can be carried out.

The analyte is preferably an amino acid, peptide, polypeptide or a protein. The amino acid, peptide, polypeptide or protein can be naturally-occurring or non-naturally-occurring. The polypeptide or protein can include within it synthetic or modified amino acids. A number of different types of modification to amino acids are known in the art. For the purposes of the invention, it is to be understood that the analyte can be modified by any method available in the art.

The protein can be an enzyme, antibody, hormone, growth factor or growth regulatory protein, such as a cytokine. The cytokine may be selected from an interleukin, preferably IFN-1, IL-2, IL-4, IL-5, IL-6, IL-10, IL-12 or IL-13, an interferon, preferably IL-γ or other cytokines such as TNF-α. The protein may be a bacterial protein, fungal protein, virus protein or parasite-derived protein. Before it is contacted with the pore or channel, the protein may be unfolded to form a polypeptide chain. The detection of nucleotides and nucleic acids is discussed in more detail below.

The analyte is present in any suitable sample. The invention is typically carried out on a sample that is known to contain or suspected to contain the analyte. The invention may be carried out on a sample that contains one or more analytes whose identity is unknown. Alternatively, the invention may be carried out on a sample to confirm the identity of one or more analytes whose presence in the sample is known or expected.

The sample may be a biological sample. The invention may be carried out in vitro on a sample obtained from or extracted from any organism or microorganism. The organism or microorganism is typically prokaryotic or eukaryotic and typically belongs to one the five kingdoms: plantae, animalia, fungi, monera and protista. The invention may be carried out in vitro on a sample obtained from or extracted from any virus. The sample is preferably a fluid sample. The sample typically comprises a body fluid of the patient. The sample may be urine, lymph, saliva, mucus or amniotic fluid but is preferably blood, plasma or serum. Typically, the sample is human in origin, but alternatively it may be from another mammal animal such as from commercially farmed animals such as horses, cattle, sheep or pigs or may alternatively be pets such as cats or dogs.

The sample may be a non-biological sample. The non-biological sample is preferably a fluid sample. Examples of a non-biological sample include surgical fluids, water such as drinking water, sea water or river water, and reagents for laboratory tests.

The sample is typically processed prior to being assayed, for example by centrifugation or by passage through a membrane that filters out unwanted molecules or cells, such as red blood cells. The sample may be measured immediately upon being taken. The sample may also be typically stored prior to assay, preferably below −70° C.

The pore or channel typically comprises a molecular adaptor that facilitates its interaction with the analyte. The presence of the adaptor improves the host-guest chemistry of the pore or channel and analyte. The principles of host-guest chemistry are well-known in the art. The adaptor has an effect on the physical or chemical properties of the pore or channel that improves its interaction with analytes. For a pore, the adaptor typically alters the charge of the barrel or channel of the pore or specifically interacts with or binds to analytes thereby facilitating their interaction with the pore or channel. For a channel, the adaptor typically specifically interacts with or binds to analytes thereby facilitating their interaction with the channel.

The adaptor mediates the interaction between analytes and the pore or channel. The analytes preferably reversibly bind to the pore via or in conjunction with the adaptor.

In the case of pores, the analyte most preferably reversibly binds to the pore via or in conjunction with the adaptor as it passes through the pore across the lipid bilayer. The analyte can also reversibly bind to the barrel or channel of the pore via or in conjunction with the adaptor as it passes through the pore across the lipid bilayer. The adaptor preferably constricts the barrel or channel so that it may interact with the analytes.

Suitable adaptors for channels are well known in art (e.g. Bayley and Cremer, Nature 413, 226-230 (2001); and Chen et al., Proc. Natl. Acad. Sci. USA 105, 6272-6277 (2008)). For pores, the adaptor is typically cyclic. The adaptor preferably has the same symmetry as the pore. An adaptor having seven-fold symmetry is typically used if the pore is heptameric (e.g. has seven subunits around a central axis that contribute 14 strands to a transmembrane β barrel). Likewise, an adaptor having six-fold symmetry is typically used if the pore is hexameric (e.g. has six subunits around a central axis that contribute 12 strands to a transmembrane β barrel, or is a 12-stranded β barrel). Any adaptor that facilitates the interaction between the pore or channel and the analyte can be used. Suitable adaptors include, but are not limited to, cyclodextrins, cyclic peptides and cucurbiturils. The adaptor is preferably a cyclodextrin or a derivative thereof. The adaptor is more preferably heptakis-6-amino-β-cyclodextrin (am₇-βCD), 6-monodeoxy-6-monoamino-β-cyclodextrin (am₁-βCD) or heptakis-(6-deoxy-6-guanidino)-cyclodextrin (gu₇-βCD). Table 5 below shows preferred combinations of pores and adaptors.

TABLE 5
Suitable combinations of pores and adaptors

| | Number of strands in | |
|---|---|---|
| | the transmembrane | |
| Pore | β-barrel | Adaptor |
| Leukocidin | 16 | γ-cyclodextrin (γ-CD) |
| OmpF | 16 | γ-cyclodextrin (γ-CD) |
| α-hemolysin (or a | 14 | β-cyclodextrin (β-CD) |
| variant thereof | | 6-monodeoxy-6- |
| discussed above) | | monoamino-β-cyclodextrin |
| | | (am₁β-CD) |
| | | heptakis-6-amino-β- |
| | | cyclodextrin (am₇-β-CD) |
| | | heptakis-(6-deoxy-6- |
| | | guanidino)-cyclodextrin |
| | | (gu₇-β-CD) |
| OmpG | 14 | β-cyclodextrin (β-CD) |
| | | 6-monodeoxy-6- |
| | | monoamino-β-cyclodextrin |
| | | (am₁β-CD) |
| | | heptakis-6-amino-β- |
| | | cyclodextrin (am₇-β-CD) |
| | | heptakis-(6-deoxy-6- |
| | | guanidino)-cyclodextrin |
| | | (gu₇-β-CD) |
| NalP | 12 | α-cyclodextrin (α-CD) |
| OMPLA | 12 | α-cyclodextrin (α-CD) |

The adaptor is preferably covalently attached to the pore or channel. The adaptor can be covalently attached to the pore or channel using any method known in the art. The adaptor may be attached directly to the pore or channel. The adaptor is preferably attached to the pore or channel using a bifunctional crosslinker. Suitable crosslinkers are well-known in the art. Preferred crosslinkers include 2,5-dioxopyrrolidin-1-yl 3-(pyridin-2-yldisulfanyl)propanoate, 2,5-dioxopyrrolidin-1-yl 4-(pyridin-2-yldisulfanyl)butanoate and 2,5-dioxopyrrolidin-1-yl 8-(pyridin-2-yldisulfanyl)octananoate. The most preferred crosslinker is succinimidyl 3-(2-pyridyldithio)propionate (SPDP). Typically, the adaptor is covalently attached to the bifunctional crosslinker before the adaptor/crosslinker complex is covalently attached to the pore or channel but it is also possible to covalently attach the bifunctional crosslinker to the pore or channel before the bifunctional crosslinker/pore complex is attached to the adaptor.

The site of covalent attachment is selected such that the adaptor facilitates interaction of analytes with the pore or channel and thereby allows detection of analytes. For pores based on α-HL, the correct orientation of the adaptor within the barrel or channel of the pore and the covalent attachment of adaptor to the pore can be facilitated using specific modifications to the pore. In particular, every subunit of the pore preferably has a glutamine at position 139 of SEQ ID NO: 2. One or more of the subunits of the pore may have an arginine at position 113 of SEQ ID NO: 2. One or more of the subunits of the pore may have a cysteine at position 119, 121 or 135 of SEQ ID NO: 2 to facilitate attachment of the molecular adaptor to the pore.

The analyte may be contacted with the pore or channel on either side of the lipid bilayer. The analyte may be introduced to the pore or channel on either side of the lipid bilayer. The analyte may be contacted with the side of the lipid bilayer that allows the analyte to pass through the pore to the other side of the lipid bilayer. For example, the analyte is contacted with an end of the pore, which in its native environment allows the entry of ions or small molecules, such as analytes, into the barrel or channel of the pore such that the analyte may pass through the pore. In such cases, the analyte interacts with the pore and/or adaptor as it passes across the lipid bilayer through the barrel or channel of the pore. Alternatively, the analyte may be contacted with the side of the lipid bilayer that allows the analyte to interact with the pore or channel via or in conjunction with the adaptor, dissociate from the pore or channel and remain on the same side of the lipid bilayer. Pores or channels in which the orientation of the adaptor is fixed may be used. As a result, the analyte is preferably contacted with the end of the pore or channel towards which the adaptor is oriented. Most preferably, the analyte is contacted with the end of the pore or channel towards which the portion of the adaptor that interacts with the analyte is orientated.

### Methods of Sequencing

A lipid bilayer of the invention may be used to sequence nucleic acids. The lipid bilayer comprises at least one transmembrane pore. Any number of pores may be present in the lipid bilayer as discussed above. The lipid bilayer preferably comprises only one transmembrane pore. The transmembrane pore typically has a nucleic acid binding protein, preferably a nucleic acid handling protein, covalently attached thereto. The pore may be any of those described in International Application No. PCT/GB09/001,679 (published as WO 2010/004265) or PCT/GB10/000,133 (published as WO 2010/086603).

The nucleic acid binding protein, which is preferably a nucleic acid handling enzyme such as an exonuclease, attached to the pore handles a target nucleic acid sequence in such a way that a proportion of the nucleotide in the target sequence interacts with the pore, preferably the barrel or channel of the pore. Nucleotides are then distinguished on the basis of the different ways in which they affect the current flowing through the pore during the interaction.

Each nucleotide may be digested from one of the target sequence in a processive manner or the target sequence may be pushed or pulled through the pore. This ensures that a proportion of the nucleotides in the target nucleic acid sequence interacts with the pore and is identified. The lack of any interruption in the signal is important when sequencing nucleic acids. When the enzyme and the pore are covalently attached it means they can be stored together, thereby allowing the production of a ready-to-use sensor.

In one embodiment, an exonuclease enzyme, such as a deoxyribonuclease, is attached to the pore such that a proportion of the nucleotides is released from the target nucleic acid and interacts with the barrel or channel of the pore. In another embodiment, an enzyme that is capable of pushing or pulling the target nucleic acid sequence through the pore is optionally attached to the pore and is used such that the target nucleic acid sequence is pushed or pulled through the barrel or channel of the pore and a proportion of the nucleotides in the target sequence interacts with the barrel or channel. In this embodiment, the nucleotides may interact with the pore in blocks or groups of more than one, such as 2, 3 or 4. The nucleotides may interact with the pore one at a time. Suitable enzymes include, but are not limited to, polymerases, nucleases, helicases and topoisomerases, such as gyrases. In each embodiment, the enzyme may be attached to the pore at a site in close proximity to the opening of the barrel of channel of the pore. When the enzyme is attached to the pore it is preferably such that its active site is orientated towards the opening of the barrel of channel of the pore. This means that a proportion of the nucleotides of the target nucleic acid sequence is fed in the barrel or channel. The enzyme is preferably attached to the cis side of the pore.

The modified pore may be derived from any of the transmembrane pores discussed above, including the β-barrel pores and α-helix bundle pores.

For pores comprising the sequence shown in SEQ ID NO: 2 or a variant thereof, the pore typically comprises an appropriate number of additional subunits comprising the sequence shown in SEQ ID NO: 2 or a variant thereof. A preferred pore comprises one subunit comprising the sequence shown in SEQ ID NO: 2 or a variant thereof covalently attached to the nucleic acid binding protein and six subunits comprising the sequence shown in SEQ ID NO: 2 or a variant thereof. The pore may comprise one or more subunits comprising the sequence shown in SEQ ID NO: 4 or a variant thereof. SEQ ID NO: 4 shows the sequence of SEQ ID NO: 2 except that it has an cysteine at position 135 (L135C) and a glutamine at position 139 (N139Q). A variant of SEQ ID NO: 4 may differ from SEQ ID NO: 4 in the same way and to the same extent as discussed for SEQ ID NO: 2 above. Another preferred pore comprises one subunit comprising the sequence shown in SEQ ID NO: 2 or a variant thereof covalently attached to the nucleic acid binding protein and six subunits comprising the sequence shown in SEQ ID NO: 4 or a variant thereof.

The pore(s) may comprise a molecular adaptor that facilitates the interaction between the pore and the nucleotides or the target nucleic acid sequence. Such adaptors are discussed above with reference to stochastic sensing.

In one embodiment, the method comprises (a) contacting the target sequence with a lipid bilayer of the invention, which comprises at least one pore having a molecular adaptor and an exonuclease covalently attached thereto, such that the exonuclease digests an individual nucleotide from one end of the target sequence; (b) contacting the nucleotide with the pore so that the nucleotide interacts with the adaptor; (c) measuring the current passing through the pore during the interaction and thereby determining the identity of the nucleotide; and (d) repeating steps (a) to (c) at the same end of the target sequence and thereby determining the sequence of the target sequence. Hence, the method involves stochastic sensing of a proportion of the nucleotides in a target nucleic acid sequence in a successive manner in order to sequence the target sequence. An individual nucleotide is a single nucleotide. An individual nucleotide is one which is not bound to another nucleotide or nucleic acid by any bond, such as a phosphodiester bond. A phosphodiester bond involves one of the phosphate groups of a nucleotide being bound to the sugar group of another nucleotide. An individual nucleotide is typically one which is not bound in any manner to another nucleic acid sequence of at least 5, at least 10, at least 20, at least 50, at least 100, at least 200, at least 500, at least 1000 or at least 5000 nucleotides.

In another embodiment, the method comprises (a) contacting the target sequence with a lipid bilayer of the invention, which comprises at least one pore having a molecular adaptor and a nucleic acid binding protein covalently attached thereto, so that the target sequence is pushed or pulled through the pore and a proportion of the nucleotides in the target sequence interacts with the pore and (b) measuring the current passing through the pore during each interaction and thereby determining the sequence of the target sequence. In another embodiment, the method comprises (a) contacting the target sequence with a lipid bilayer of the invention, which comprises at least one transmembrane pore so that the target sequence translocates through the pore and a proportion of the nucleotides in the target sequence interacts with the pore and (b) measuring the current passing through the pore during each interaction and thereby determining the sequence of the target sequence. Hence, the method involves stochastic sensing of a proportion of the nucleotides in a target nucleic acid sequence as the nucleotides pass through the barrel or channel in a successive manner in order to sequence the target sequence.

The whole or only part of the target nucleic acid sequence may be sequenced using this method. The nucleic acid sequence can be any length. For example, the nucleic acid sequence can be at least 10, at least 50, at least 100, at least 150, at least 200, at least 250, at least 300, at least 400 or at least 500 nucleotides in length. The nucleic acid sequence can be naturally occurring or artificial. For instance, the method may be used to verify the sequence of a manufactured oligonucleotide. The methods are typically carried out in vitro.

### Interaction Between the Pore and Nucleotides

The nucleotide or nucleic acid may be contacted with the pore on either side of the lipid bilayer. The nucleotide or nucleic acid may be introduced to the pore on either side of the lipid bilayer. The nucleotide or nucleic acid is typically contacted with the side of the lipid bilayer on which the enzyme is attached to the pore. This allows the enzyme to handle the nucleic acid during the method.

A proportion of the nucleotides of the target nucleic acid sequence interacts with the pore and/or adaptor as it passes across the lipid bilayer through the barrel or channel of the pore. Alternatively, if the target sequence is digested by an exonuclease, the nucleotide may interact with the pore via or in conjunction with the adaptor, dissociate from the pore and remain on the same side of the lipid bilayer. The methods may involve the use of pores in which the orientation of the adaptor is fixed. In such embodiments, the nucleotide is preferably contacted with the end of the pore towards which the adaptor is oriented. Most preferably, the nucleotide is contacted with the end of the pore towards which the portion of the adaptor that interacts with the nucleotide is orientated.

The nucleotides may interact with the pore in any manner and at any site. As discussed above, the nucleotides preferably reversibly bind to the pore via or in conjunction with the adaptor. The nucleotides most preferably reversibly bind to the pore via or in conjunction with the adaptor as they pass through the pore across the lipid bilayer. The nucleotides can also reversibly bind to the barrel or channel of the pore via or in conjunction with the adaptor as they pass through the pore across the lipid bilayer.

During the interaction between a nucleotide and the pore, the nucleotide affects the current flowing through the pore in a manner specific for that nucleotide. For example, a particular nucleotide will reduce the current flowing through the pore for a particular mean time period and to a particular extent. In other words, the current flowing through the pore is distinctive for a particular nucleotide. Control experiments may be carried out to determine the effect a particular nucleotide has on the current flowing through the pore. Results from carrying out the method of the invention on a test sample can then be compared with those derived from such a control experiment in order to identify a particular nucleotide.

### Apparatus

The methods may be carried out using any apparatus that is suitable for investigating a lipid bilayer/pore system comprising a lipid bilayer of the invention. The methods may be carried out using any apparatus that is suitable for stochastic sensing. For example, the apparatus comprises a chamber comprising an aqueous solution and a barrier that separates the chamber into two sections. The barrier has an aperture in which the lipid bilayer containing the pore is formed. The nucleotide or nucleic acid may be contacted with the pore by introducing the nucleic acid into the chamber. The nucleic acid may be introduced into either of the two sections of the chamber, but is preferably introduced into the section of the chamber containing the enzyme.

The methods may be carried out using the apparatus described in International Application No. PCT/GB08/000562.

The methods involve measuring the current passing through the pore during interaction with the nucleotides. Therefore the apparatus also comprises an electrical circuit capable of applying a potential and measuring an electrical signal across the lipid bilayer and pore. The methods may be carried out using a patch clamp or a voltage clamp. The methods preferably involve the use of a voltage clamp.

### Conditions

The methods of the invention involve the measuring of a current passing through the pore during interaction with nucleotides of a target nucleic acid sequence. Suitable conditions for measuring ionic currents through transmembrane pores are known in the art and disclosed in the Examples. The method is carried out with a voltage applied across the lipid bilayer and pore. The voltage used is typically from −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 120 mV to 170 mV. It is possible to increase discrimination between different nucleotides by a pore of the invention by varying the applied potential.

The methods are carried out in the presence of any alkali metal chloride salt. In the exemplary apparatus discussed above, the salt is present in the aqueous solution in the chamber. Potassium chloride (KCl), sodium chloride (NaCl) or caesium chloride (CsCl) is typically used. KCl is preferred. The salt concentration is typically from 0.1 to 2.5M, from 0.3 to 1.9M, from 0.5 to 1.8M, from 0.7 to 1.7M, from 0.9 to 1.6M or from 1M to 1.4M. High salt concentrations provide a high signal to noise ratio and allow for currents indicative of the presence of a nucleotide to be identified against the background of normal current fluctuations. However, lower salt concentrations may have to be used so that the enzyme is capable of functioning.

The methods are typically carried out in the presence of a buffer. In the exemplary apparatus discussed above, the buffer is present in the aqueous solution in the chamber. Any buffer may be used in the methods. One suitable buffer is Tris-HCl buffer. The methods are typically carried out at a pH of from 4.0 to 10.0, from 4.5 to 9.5, from 5.0 to 9.0, from 5.5 to 8.8, from 6.0 to 8.7 or from 7.0 to 8.8 or 7.5 to 8.5. The pH used is preferably about 7.5.

The methods are typically carried out at from 0° C. to 100° C., from 15° C. to 95° C., from 16° C. to 90° C., from 17° C. to 85° C., from 18° C. to 80° C., 19° C. to 70° C., or from 20° C. to 60° C. The methods may be carried out at room temperature. The methods are preferably carried out at a temperature that supports enzyme function, such as about 37° C. Good nucleotide discrimination can be achieved at low salt concentrations if the temperature is increased. However, lower temperatures, particularly those below room temperature, result in longer dwell times and can therefore be used to obtain a higher degree of accuracy.

In addition to increasing the solution temperature, there are a number of other strategies that can be employed to increase the conductance of the solution, while maintaining conditions that are suitable for enzyme activity. One such strategy is to use the lipid bilayer to divide two different concentrations of salt solution, a low salt concentration of salt on the enzyme side and a higher concentration on the opposite side. One example of this approach is to use 200 mM of KCl on the cis side of the lipid bilayer and 500 mM KCl in the trans chamber. At these conditions, the conductance through the pore is expected to be roughly equivalent to 400 mM KCl under normal conditions, and the enzyme only experiences 200 mM if placed on the cis side. Another possible benefit of using asymmetric salt conditions is the osmotic gradient induced across the pore. This net flow of water could be used to pull nucleotides into the pore for detection. A similar effect can be achieved using a neutral osmolyte, such as sucrose, glycerol or PEG. Another possibility is to use a solution with relatively low levels of KCl and rely on an additional charge carrying species that is less disruptive to enzyme activity.

### Exonuclease-Based Methods

In one embodiment, the method of sequencing a target nucleic acid sequence involves contacting the target sequence with a pore having an exonuclease enzyme, such as deoxyribonuclease, attached thereto. Any of the exonuclease enzymes discussed above may be used in the method. The exonuclease releases individual nucleotides from one end of the target sequence. Exonucleases are enzymes that typically latch onto one end of a nucleic acid sequence and digest the sequence one nucleotide at a time from that end. The exonuclease can digest the nucleic acid in the 5′ to 3′ direction or 3′ to 5′ direction. The end of the nucleic acid to which the exonuclease binds is typically determined through the choice of enzyme used and/or using methods known in the art. Hydroxyl groups or cap structures at either end of the nucleic acid sequence may typically be used to prevent or facilitate the binding of the exonuclease to a particular end of the nucleic acid sequence.

The method involves contacting the nucleic acid sequence with the exonuclease so that the nucleotides are digested from the end of the nucleic acid at a rate that allows identification of a proportion of nucleotides as discussed above. Methods for doing this are well known in the art. For example, Edman degradation is used to successively digest single amino acids from the end of polypeptide such that they may be identified using High Performance Liquid Chromatography (HPLC). A homologous method may be used in the invention.

The rate at which the exonuclease functions can be altered by mutation compared to the wild type enzyme. A suitable rate of activity of the exonuclease in the method of sequencing involves digestion of from 0.5 to 1000 nucleotides per second, from 0.6 to 500 nucleotides per second, 0.7 to 200 nucleotides per second, from 0.8 to 100 nucleotides per second, from 0.9 to 50 nucleotides per second or 1 to 20 or 10 nucleotides per second. The rate is preferably 1, 10, 100, 500 or 1000 nucleotides per second. A suitable rate of exonuclease activity can be achieved in various ways. For example, variant exonucleases with a reduced or improved optimal rate of activity may be used in accordance with the invention.

### Pushing or Pulling DNA Through the Pore

Strand sequencing involves the controlled and stepwise translocation of nucleic acid polymers through a pore. The majority of DNA handling enzymes are suitable for use in this application provided they hydrolyse, polymerise or process single stranded DNA or RNA. Preferred enzymes are polymerases, nucleases, helicases and topoisomerases, such as gyrases. The enzyme moiety is not required to be in as close a proximity to the pore lumen as for individual nucleotide sequencing as there is no potential for disorder in the series in which nucleotides reach the sensing moiety of the pore.

The two strategies for single strand DNA sequencing are the translocation of the DNA through the nanopore, both cis to trans and trans to cis, either with or against an applied potential. The most advantageous mechanism for strand sequencing is the controlled translocation of single strand DNA through the nanopore with an applied potential. Exonucleases that act progressively or processively on double stranded DNA can be used on the cis side of the pore to feed the remaining single strand through under an applied potential or the trans side under a reverse potential. Likewise, a helicase that unwinds the double stranded DNA can also be used in a similar manner. There are also possibilities for sequencing applications that require strand translocation against an applied potential, but the DNA must be first “caught” by the enzyme under a reverse or no potential. With the potential then switched back following binding the strand will pass cis to trans through the pore and be held in an extended conformation by the current flow. The single strand DNA exonucleases or single strand DNA dependent polymerases can act as molecular motors to pull the recently translocated single strand back through the pore in a controlled stepwise manner, trans to cis, against the applied potential.

### Kits

The invention also provides kits for inserting a pre-determined number of membrane proteins into a lipid bilayer comprising (a) one or more membrane proteins and (b) a fluorinated amphiphile, wherein the membrane proteins are derived from α-hemolysin (α-HL), MspA from Mycobacterium smegmatis or Kcv of chlorella virus PBCV-1. The kits may comprise any number of membrane proteins, preferably 1, 2, 4, 5, 7, 8, 10, 12, 14, 15 or more. The kits may comprise any of the membrane proteins discussed above. A preferred kit comprises seven subunit each comprising the sequence shown in SEQ ID NO: 2 or a variant thereof. A more preferred kit comprises (i) a subunit comprising the sequence shown in SEQ ID NO: 2 or a variant thereof having a nucleic acid binding protein covalently attached thereto and (ii) six subunits each comprising the sequence shown in SEQ ID NO: 2 or a variant thereof. Other preferred kits comprise eight subunits each comprising the sequence shown in SEQ ID NO: 14 or a variant thereof or four subunits each comprising the sequence shown in SEQ ID NO: 16 or a variant thereof. The kits may comprise any of the F-amphiphiles discussed above.

The kits of the invention may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotide sequences, a lipid bilayer as defined above or voltage or patch clamp apparatus. Reagents may be present in the kit in a dry state such that a fluid sample resuspends the reagents. The kit may also, optionally, comprise instructions to enable the kit to be used in the method of the invention or details regarding which patients the method may be used for. The kit may, optionally, comprise nucleotides.

The following Example illustrates the invention:

### Example

Abbreviations: α-HL: α-hemolysin; βCD: β-cyclodextrin; CF: 5,(6)-carboxyfluorescein; F₆TAC: C₆F₁₃C₂H₄—S-poly[tris(hydroxymethyl)aminomethane]; CMC: critical micelle concentration; DPhPC: 1,2-diphytanoyl-sn-glycero-3-phosphocholine; F-amphiphile: fluorinated amphiphile; F₆OM: fluorinated octyl maltoside; F₆FC: fluorinated fos-choline; HEPES: 4-(2-hydroxyethyl)piperazine-1-ethanesulfonic acid; HFTAC: C₂H₅C₆F₁₂C₂H₄—S-poly[tris(hydroxymethyl)-aminomethane]; IVTT: in vitro transcription and translation; LUV: large unilamellar vesicles; MOPS: 3-(N-morpholino)propanesulfonic acid; rRBC: rabbit erythrocytes; WT: wild-type.

### 1 MATERIALS AND METHODS

### 1.1 Materials

Fluorinated fos-choline (F₆FC) and fluorinated octyl maltoside (F₆OM) (FIG. 1) were obtained from Anatrace. C₆F₁₃C₂H₄—S-poly[tris(hydroxymethyl)aminomethane] (F₆TAC) was a gift from the laboratory of Prof. Bernard Pucci. Stock solutions were made in water: F₆FC (100 mM), F₆OM (20 mM), F₆TAC (30 mM).

### 1.2 Protein Purification

Heptameric WT-α-HL from Staphylococcus aureus (Wood 46 strain) in SDS buffer (20 mM Na phosphate, 150 mM NaCl, 0.3% w/v SDS, at pH 8.0), for liposome assays and planar bilayer recordings, was obtained as previously reported (Maglia et al. (2009) Nano Letters 9, 3831-3836). Monomeric WT-α-HL, also for liposome assays and planar bilayer recordings, was also obtained from S. aureus by a modification of a previously reported protocol (Maglia et al. supra). After elution from the S-Sepharose FF XK-16 cation exchange column, the peak fractions containing the monomer were collected and concentrated to approximately 3 mg mL⁻¹ by using ultracentrifugal filter devices with a 10 kDa cut off (no. 4321, Amicon) spun at 2900×g. The protein concentration was determined from the absorbance at 280 nm (the OD₂₈₀ of a 1.0 mg mL⁻¹ solution is 1.95). The monomer was purified further by chromatography on a Superdex 200 HiLoad gel filtration column (no. 17107101, GE Healthcare), which was equilibrated and run with 10 mM Tris.HCl, 150 mM NaCl, pH 8.0, at a flow rate of 1 mL min⁻¹. The peak fractions were located by SDS-PAGE, pooled and concentrated to 1 mg mL⁻¹. The yield of monomer of about 95% purity, as judged by SDS-PAGE, was around 10 mg per liter of culture.

The monomeric WT-α-HL used in the hemolytic assay was expressed in an E. coli in vitro transcription and translation (IVTT) system (E. coli T7 S30 Extract System for Circular DNA, Promega) for 1 h at 37° C. with the complete amino acid mix. The solution was spun at 25,000×g for 15 min at 4° C. and the supernatant containing the α-HL monomers was retained.

The MspA (NNNRRK) mutant (GeneScript) was expressed in the IVTT system (50 μL) for 2 h at 37° C. in the presence of rRBCM (2 μL) and [³⁵S]methionine. The membranes were recovered by centrifugation and solubilized in sample buffer. The proteins were then separated in an 8% SDS-polyacrylamide gel. The gel was dried without heating onto paper (Whatman 3M) under a vacuum and the MspA oligomer band was located by autoradiography. After rehydration in buffer (300 μL of 25 mM Tris.HCl, pH 8.0), the paper was removed. The gel was crushed using a pestle and the slurry filtered through a QIAshredder column (Qiagen) by centrifugation at 25,000×g for 10 min.

The tetrameric potassium channel, Kcv, used for single channel recording experiments was obtained after IVTT in the presence of [³⁵S]methionine as previously described (Heron et al. (2007) J Am Chem Soc 129, 16042-16047).

### 1.3 Dye Leakage Assay

A 100 mM stock solution of 5,(6)-carboxyfluorescein (CF, Sigma Aldrich) was made in 150 mM NaCl, 2.7 mM KCl, 10 mM Na₂HPO₄, 2 mM KH₂PO₄, pH 7.4. The pH of the dye solution was re-adjusted to pH 7.4 with NaOH. Liposomes containing CF were made by extrusion. Soybean lecithin (20 mg, Calbiochem) was dissolved in chloroform (1 mL) in a round bottom flask. The lipid was dried under N₂ to form a thin uniform layer and further dehydrated in a vacuum desiccator for 2 to 3 h. The lipid was then slowly rehydrated by resuspension in CF solution that had been diluted 50% with water (total volume 300 μL) to give a final concentration of 50 mM dye. The flask was vortexed for a few minutes to ensure complete resuspension of the lipids, followed by five freeze-thaw cycles (liquid nitrogen/37° C. water bath). The suspension was then extruded twenty times through a 0.1 μm polycarbonate filter by using a mini-extruder (Avanti Polar Lipids) to yield large, unilamellar vesicles (LUV). Free CF was removed from the LUV suspension by size exclusion chromatography on a Sephadex G50 (Sigma Aldrich) column (1 cm×20 cm), equilibrated and eluted with 300 mM NaCl, 2.7 mM KCl, 10 mM Na₂HPO₄, 2 mM KH₂PO₄, pH 7.4 (buffer A). Before dye-release experiments, the freshly prepared liposome stock was diluted 50-fold in buffer A so that the maximal released CF fluorescence was within the range of the fluorimeter.

The release of CF, which is self-quenched within the liposomes, was assessed from the increase in fluorescence emission at 520 nm (excitation at 492 nm). At the end of each run, Triton X-100 was added to the cuvette (0.1% final concentration) to determine the maximal CF fluorescence. To determine the effects of the F-amphiphiles, α-HL was added to buffer A containing the diluted LUV and an F-amphiphile. The final volume in the cuvette was 1 mL and the amount of heptamer or monomer was 20 μg or 5 μg, respectively. The data were analyzed with Origin. The released CF as a percentage of the total at a time t is given by:

R(t)=100×[I(t)−I(0)]/[I(∞)−I(0)]

where the fluorescence intensity at time t is I(t), the initial fluorescence of the liposomes is I(0), and the fluorescence after Triton X-100 addition is I(∞).

### 1.4 Single Channel Recordings—General

The apparatus for planar lipid bilayer recording consisted of two compartments separated by a Teflon septum containing an aperture of 100 μm diameter. The aperture was pre-treated with a solution of hexadecane in pentane (10% v/v). For recordings with α-HL, buffer A was used. For Kcv, the buffer was 200 mM KCl, 10 mM HEPES, pH 7.0. To form a bilayer, buffer was added to both compartments at a level below the aperture and 1,2-diphytanoyl-sn-glycero-3-phosphocholine (DPhPC, Avanti Polar Lipids) in pentane (10 mg mL⁻¹) was added to each surface. The pentane evaporates to leave a lipid monolayer at the air-water interface. Sequential raising of the levels of the buffer solutions folds the two monolayers together to form a vertical lipid bilayer. The formation and quality of the bilayers were monitored by capacitance measurements. Currents were recorded under voltage-clamp conditions with Ag/AgCl electrodes.

1.5 Single Channel Recordings with F-Amphiphiles.

Control experiments without F-amphiphiles were initiated by the addition of α-HL heptamer or monomer (final concentration of 30 ng mL⁻¹) or Kcv (to 4 ng mL⁻¹) to the cis compartment (ground), which was stirred until insertion occurred. Experiments to examine the inhibitory effects of F-amphiphiles on the insertion of WT-α-HL or Kcv were initiated by the addition of protein to the cis compartment, which contained (1000-V) μL of electrolyte, where V was the volume of the F-amphiphile later added to the cis side after channel insertion had occurred. The cis compartment was stirred briefly after the addition of the F-amphiphile solution. To compensate for the addition of F-amphiphile solution, an equal volume of water was added to the trans compartment. The low-pass Bessel filter of the amplifier was set at 1 kHz. Data were acquired at a sampling rate of 10 kHz. The data points for the IV curves, for both WT-α-HL heptamer and Kcv are the mean values from 3 separate single channel experiments. The current values obtained in the presence of 10 mM F₆FC (cis) were normalized to the current values obtained in the absence of F₆FC to take into account the reduction in the ionic strength of the recording buffer caused by the addition of the F-amphiphile.

### 1.6 Single Channel Recordings-β-Cyclodextrin Binding Studies

The recording buffer contained 1 M NaCl and 10 mM Na₂HPO₄, adjusted to pH 7.5 with aqueous HCl. The experiments were initiated by the addition of ˜30 ng WT-α-HL heptamer to the cis compartment with stirring until a channel inserted. βCD (40 μM) was added to the trans compartment. The internal low-pass Bessel filter of the amplifier was set at 5 kHz and the acquisition rate was 20 kHz. τₒₙ and τ_{off} values for βCD binding to the WT-α-HL pore, at ±40 mV, were obtained from dwell-time histograms fitted to single exponentials with the Clampfit software. To determine kinetic constants for the association and dissociation of βCD in the presence of the F-amphiphiles, data from three separate single channel experiments were averaged.

### 1.7 Hemolytic Assay in Presence of F-Amphiphiles

WT-α-HL (5 μL of IVTT protein), incubated for 10 min with either 10 mM F₆FC, 5 mM F₆OM or 2 mM F₆TAC in a final volume of 10 μL, was diluted into MBSA (10 mM MOPS, 150 mM NaCl, pH 7.4, containing 1 mg mL⁻¹ bovine serum albumin) in the first well of a microtiter plate (final volume, 100 μL). The protein was then subjected to serial two-fold dilution in MBSA over the remaining 11 wells of the plate row (final volumes, 50 μL). An equal volume of 1% washed rabbit erythrocytes (rRBC) in MBSA was then added to each well, beginning with the most diluted lane. Hemolysis was followed for 1.5 h at 24° C. by monitoring the decrease in light scattering at 595 nm with a Bio-Rad microplate reader (model 3550-UV) running Microplate Manager 4.0 software.

### 2 RESULTS AND DISCUSSION

### 2.1 Effects of F-Amphiphiles on Membranes

Three different F-amphiphiles were tested in this study (FIG. 1): F-fos-choline (F₆FC) with a zwitterionic headgroup, F-octyl maltoside (F₆OM) with a non-ionic disaccharide headgroup and C₆F₁₃C₂H₄—S-poly[tris(hydroxymethyl)aminomethane] (F₆TAC) with a non-ionic polymeric headgroup (Park et al. (2007) Biochem J 403, 183-187). Earlier work suggested that F₆TAC neither solubilizes lipid bilayers nor interferes with protein synthesis at up to 50 times its CMC. In the present work, F₆FC, F₆OM and F₆TAC were tested for their effects on membranes in a hemolytic assay (Park et al. (2007) Biochem J 403, 183-187) and on planar lipid bilayers. All three F-amphiphiles lacked lytic activity towards 1% rRBCs at concentrations of up to 50 times the CMC (data not shown). In accordance with previous reports (Park et al. (2007) Biochem J 403, 183-187), F₆FC and F₆TAC did not affect the stability of pre-formed planar lipid bilayers, as determined by capacitance measurements, even at concentrations of 5 times the CMC. However, the addition of F₆OM at concentrations above the CMC did decrease the stability of pre-formed bilayers, reducing the lifetime to less than 2 min (data not shown). Therefore, electrical recordings with planar bilayers were confined to F₆FC and F₆TAC. It should also be noted that we were unable to form or reform bilayers in the presence of any one of the three F-amphiphiles.

2.2 Effects of F-Amphiphiles on the Insertion of α-HL into Liposomes

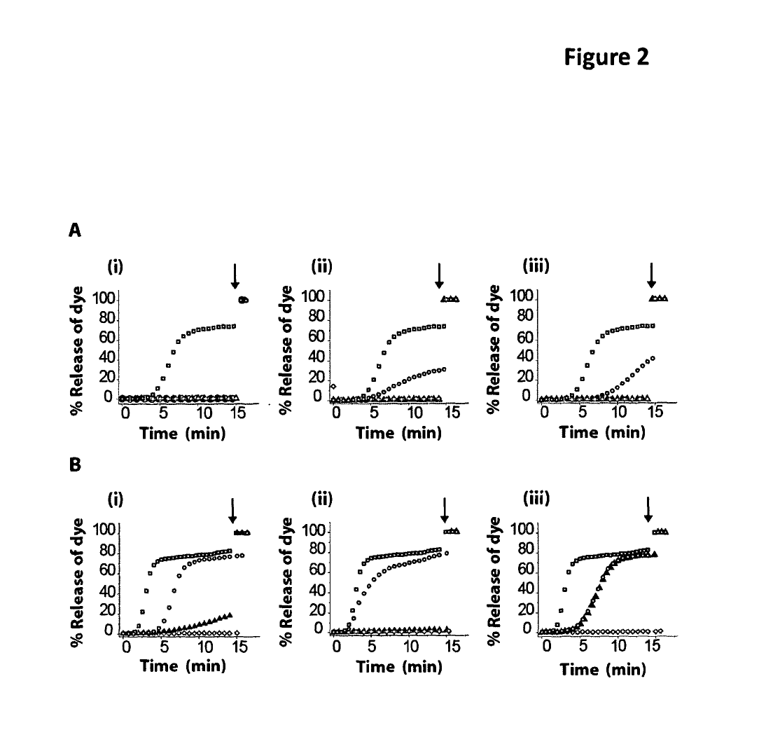

α-HL is a pore-forming protein capable of transporting molecules as large as 2 to 4 kDa through lipid bilayers. Therefore, a dye leakage assay was chosen to monitor the incorporation of α-HL into liposome (LUV) membranes. 5,(6)-Carboxyfluorescein (CF) was incorporated into LUV at self-quenching concentrations (Weinstein et al. (1977) Science 195, 489-492). When pores are formed in the LUV, dye leakage occurs, generating a fluorescence signal. At the end of the assay, the maximal fluorescence signal is measured after lysing the liposomes with detergent. We measured the lag phase before dye release, the initial rate of dye release and the final extent of release as a percentage of the maximal signal (FIG. 2, Table 6).

TABLE 6
Carboxyfluorescein release from liposomes induced by α-HL monomers and
heptamers in the absence and presence of F-amphiphiles

| | | | | ^{b}Initial | | ^{c}Lag time before | |
|---|---|---|---|---|---|---|---|
| | | ^{a}Percent release of | | rate of release | | initiation of release | |
| | | dye at end point (%) | | of dye (% min⁻¹) | | (min) | |
| | | Monomer | Heptamer | Monomer | Heptamer | Monomer | Heptamer |
| No addition of | | 74 | 84 | 18 | 37 | 3.7 | 1.6 |
| F-amphiphile | | | | | | | |
| F₆FC | Above | 0.2 | 0.6 | n.d^{e} | n.d^{e} | n.d^{e} | n.d^{e} |
| | CMC^{d} | | | | | | |
| | At | 0.2 | 20 | n.d^{e} | 2 | n.d^{e} | 2.8 |
| | CMC^{d} | | | | | | |
| | Below | 0.3 | 78 | n.d^{e} | 23 | n.d^{e} | 4.3 |
| | CMC^{d} | | | | | | |
| F₆OM | Above | 1 | 1 | n.d^{e} | n.d^{e} | n.d^{e} | n.d^{e} |
| | CMC^{d} | | | | | | |
| | At | 1 | 2.5 | n.d^{e} | n.d^{e} | n.d^{e} | n.d^{e} |
| | CMC^{d} | | | | | | |
| | Below | 31 | 79 | 4 | 16 | 4.4 | 1.8 |
| | CMC^{d} | | | | | | |
| F₆TAC | Above | 0.5 | 0.7 | n.d^{e} | n.d^{e} | n.d^{e} | n.d^{e} |
| | CMC^{d} | | | | | | |
| | At | 0.7 | 77 | n.d^{e} | 16 | n.d^{e} | 4.8 |
| | CMC^{d} | | | | | | |
| | Below | 44 | 77 | 6.5 | 16 | 7.5 | 4.8 |
| | CMC^{d} | | | | | | |

^{a}The endpoint is given as the percentage of dye released. When release was still occurring after 15 min, the percentage release at that time is given.
^{b}The initial rate of dye release was calculated from the slope of the initial linear phase.
^{c}The lag time before the initiation of dye release was determined from the intercept of the initial linear phase with the x-axis.
^{d}The three concentrations of F-amphiphile were (mM): F₆FC: 0.5, 2.0, 10; F₆OM: 0.2, 1.0, 5.0; F₆TAC: 0.05, 0.3, 2.0. The concentration of liposomes was 25 μM (in lipid monomers). The protein concentrations were: α-HL monomer, 5 μg mL⁻¹; α-HL heptamer, 20 μg mL⁻¹.
^{e}n.d. denotes not determined (in cases where there was no liposome permeabilization).
The data shown are from a typical experiment (FIG. 2).

Both the α-HL monomer and the pre-formed α-HL heptamer were examined in the presence of the F-amphiphiles, each at three concentrations: (i) below the CMC, (ii) at around the CMC, (iii) above the CMC (FIG. 2). To effect dye leakage, the α-HL monomer must both assemble and insert (Bayley, H. (2009) Nature 459, 651-652) into the LUV bilayer, while the pre-formed α-HL heptamer must undergo direct insertion from dilute detergent (Braha et al. (1997) Chem. Biol. 4, 497-505). At concentrations above the CMC, the F-amphiphiles completely prevented the action of both the α-HL monomer and the α-HL heptamer (FIG. 2). Lower concentrations of the F-amphiphiles slowed α-HL insertion to varying extents (FIG. 2). F-amphiphiles, including single chain molecules, self-assemble above the CMC to form extended structures, including tubules and structures containing bilayers such as unilamellar or multilamellar vesicles. The nature of these aggregates depends upon the concentration and structure of the amphiphile; only F-amphiphiles with bulky, branched oligosaccharide headgroups have been reported to form true micelles. Our experiments suggest that aggregates formed by F-amphiphiles prevent the insertion of both monomeric and heptameric α-HL into lipid bilayers by sequestering the proteins. However, unlike the micelles formed by standard hydrocarbon-based detergents, the F-amphiphile aggregates are unable to solubilize lipid bilayers.

2.3 Effect of F-Amphiphiles on the Insertion of Membrane Proteins into Planar Lipid Bilayers

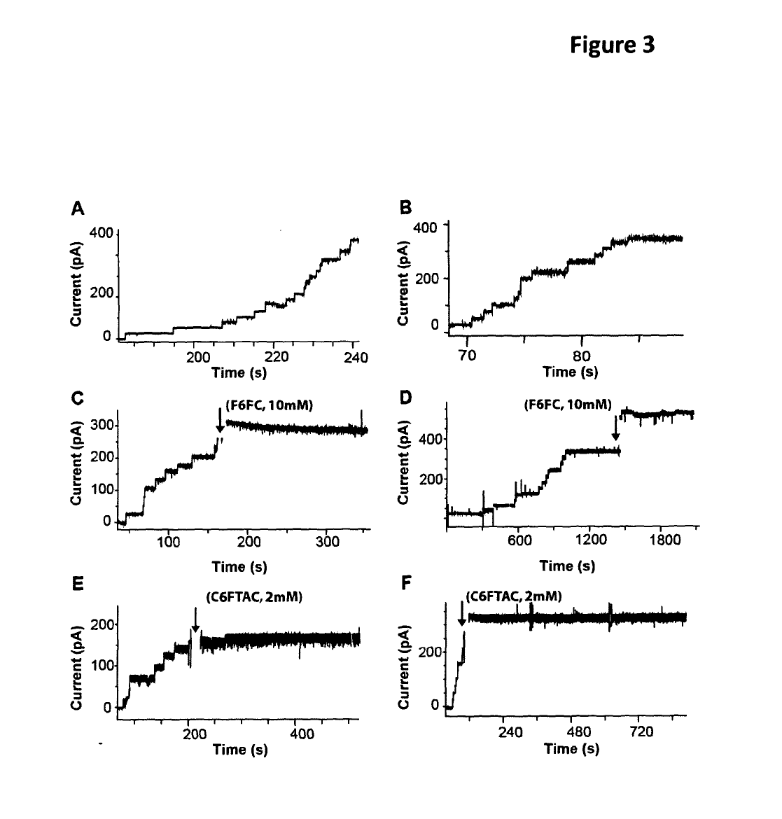

Preformed α-HL heptamers and other membrane proteins with bound detergent (SDS in the present case, see Materials and Methods) have been reported to insert directly into lipid bilayers. By contrast, α-HL monomers first oligomerize to form a heptameric pre-pore on the bilayer surface. The pre-pore then undergoes a conformational reorganization, forming a β barrel during insertion into the bilayer. In the absence of F-amphiphiles, multiple insertions of α-HL heptamers (FIG. 3A) and monomers (data not shown) were observed when these proteins were added to the cis compartment of the bilayer apparatus. When F-amphiphiles were present in the trans compartment, at final concentrations of 5 times the CMC, the rate of α-HL insertion was comparable to the rate in the absence of F-amphiphile (FIG. 3B). By contrast, both F₆FC and F₆TAC, at above the CMC in the cis compartment, caused complete arrest of the insertion of both α-HL heptamer and monomer (FIG. 3C-F). When either F₆FC or F₆TAC was present at concentrations below the CMC in the cis compartment, pore insertion events continued (data not shown).

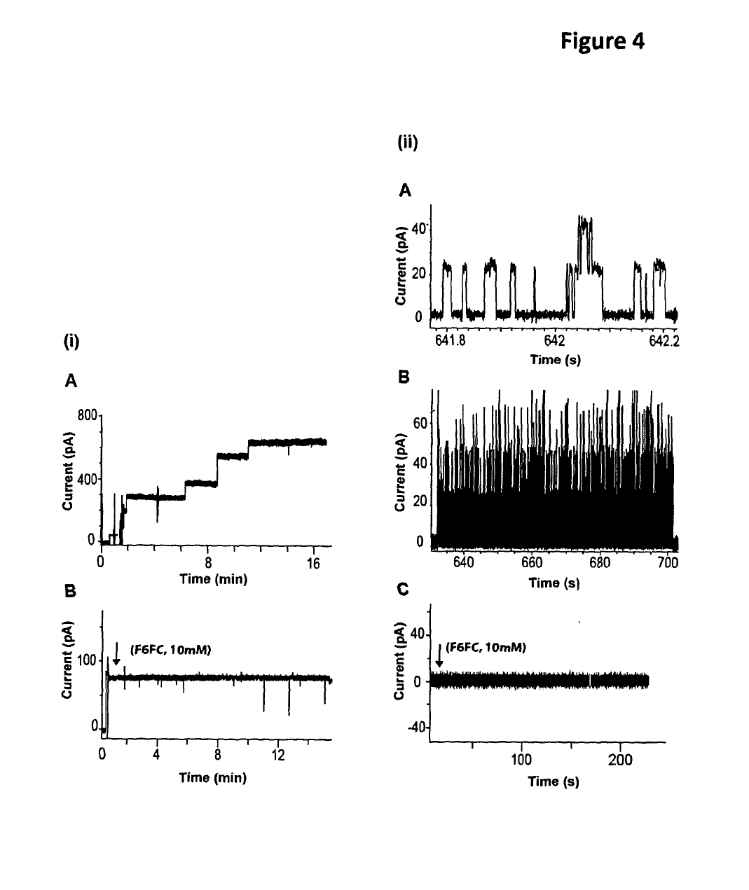

The octameric porin MspA, which is also largely β structure, behaved similarly to α-HL (FIG. 4 panel (i) A,B). Further, the α-helical membrane protein Kcv was also examined as a gel-purified tetramer. In three separate attempts, we found that no Kcv channels inserted into a bilayer when F-amphiphiles were present at concentrations above the CMC (FIG. 4 panel (ii) C), while channel insertion occurred in all three attempts in the absence of F-amphiphile (FIG. 4 panel (ii) A,B). In neither case was insertion reversed. Because both major classes of membrane protein (β barrels and α-helix bundles) are similarly affected under the conditions of our experiments, a common mechanism for the arrest of insertion must be invoked, and we favor sequestration within F-amphiphile aggregates, which are may comprise bilayer structures. These experiments show that F-amphiphiles provide a useful means to control the number of proteins entering a lipid bilayer.

### 2.4 Effect of F₆FC on α-HL Pores and Kcv Channels in Bilayers

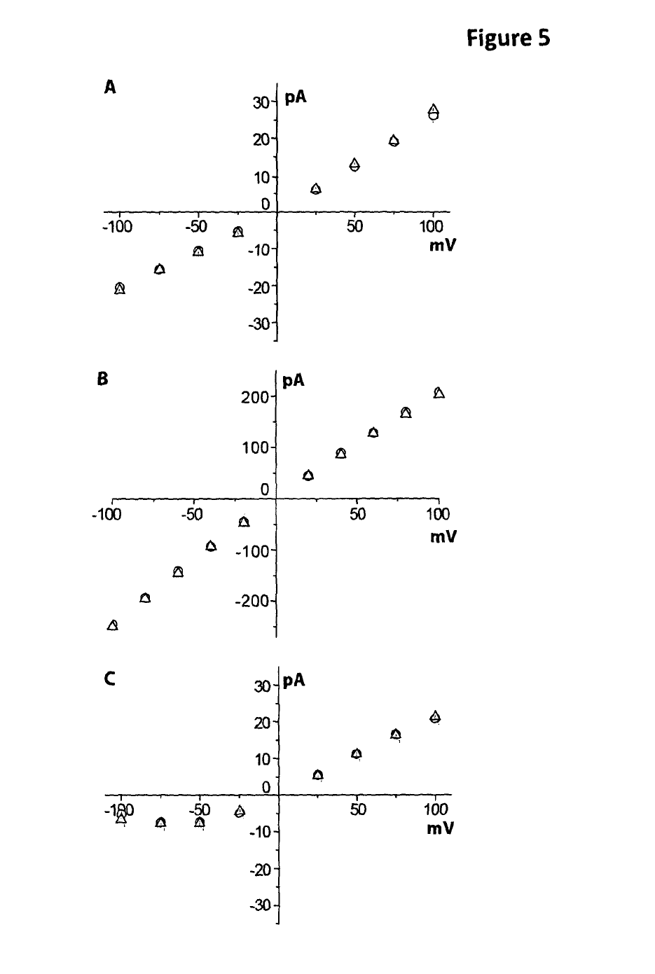

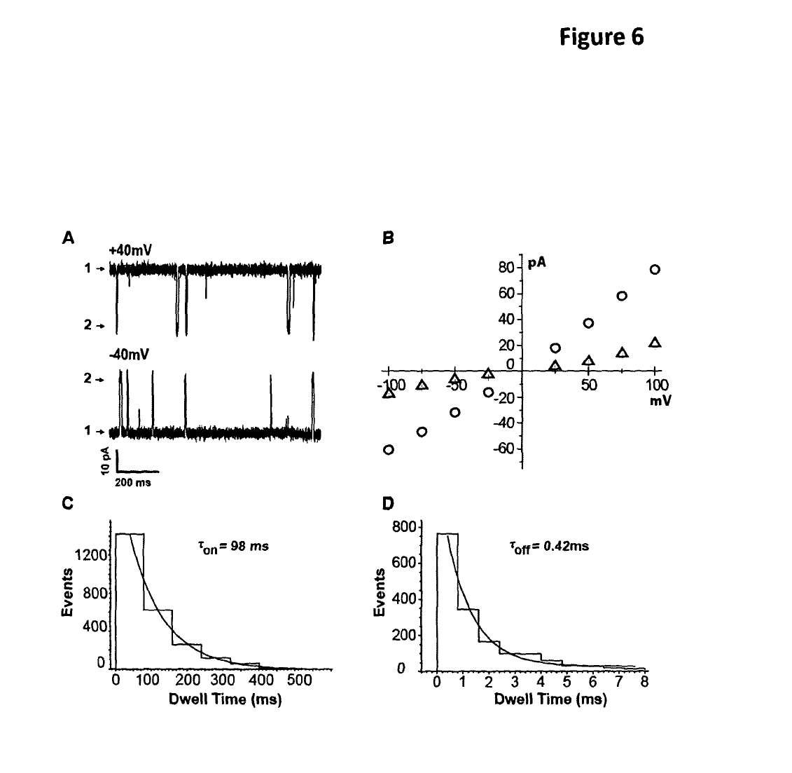

To be of genuine utility in controlling insertion, an F-amphiphile should not affect the functional properties of channels and pores that have already inserted into bilayers. Gratifyingly, the addition of F₆FC, which is commercially available, at above the CMC to the cis compartment did not cause blockades or changes in the gating of α-HL pores, MspA pores or Kcv channels that had already inserted into a bilayer. Further, the single-channel IV curves of α-HL and Kcv were unchanged (FIG. 5). We also examined the interaction of βCD (trans) with α-HL pores in the presence of F₆FC (cis) (FIG. 6). The values of kₒₙ, and k_{off}, and hence K_{d}, for βCD were comparable to previously reported values (Table 7). Therefore, although F₆FC prevents the insertion of proteins into bilayers, it does not affect the functional properties of proteins that are already in them.

TABLE 7
Binding of βCD to the α-HL pore in the presence of F₆FC: conductance values and
kinetic constants

| | Voltage | | g_{α-HL} | g_{α-HL·βCD} | | | |
|---|---|---|---|---|---|---|---|
| | (mV) | F₆FC^{b} | (pS) | (pS) | kₒₙ (M⁻¹s⁻¹) | k_{off} (s⁻¹) | K_{d} (M) |
| 1^{c} | +40 | − | 721 ± 6 | 253 ± 4 | 2.8 ± 0.2 × 10⁵ | 2.1 ± 0.2 × 10³ | 7.8 ± 0.3 × 10⁻³ |
| 2 | +40 | + | 721 ± 4 | 232 ± 4 | 2.5 ± 0.4 × 10⁵ | 2.4 ± 0.3 × 10³ | 9.6 ± 0.4 × 10⁻³ |
| 3^{c} | −40 | − | 651 ± 4 | 240 ± 3 | 4.0 ± 0.3 × 10⁵ | 1.3 ± 0.1 × 10³ | 3.4 ± 0.3 × 10⁻³ |
| 4 | −40 | + | 650 ± 3 | 257 ± 3 | 3.1 ± 0.1 × 10⁵ | 1.7 ± 0.1 × 10³ | 5.4 ± 0.5 × 10⁻³ |

^{a}g_{α-HL} and g_{α-HL.βCD} are, respectively, the unitary conductance in the absence of βCD and the unitary conductance with βCD bound in 1M NaCl, 10 mM Na₂HPO₄, pH 7.5. kₒₙ, k_{off}, and K_{d} are quoted as the mean ± SD from three experiments.
^{b}The concentration of F₆FC was 10 mM in the cis compartment.
^{c}Values taken from a previous report (Gu et al.(2000) Biophys. J. 79, 1967-1975).

### 2.5 Mode of Action

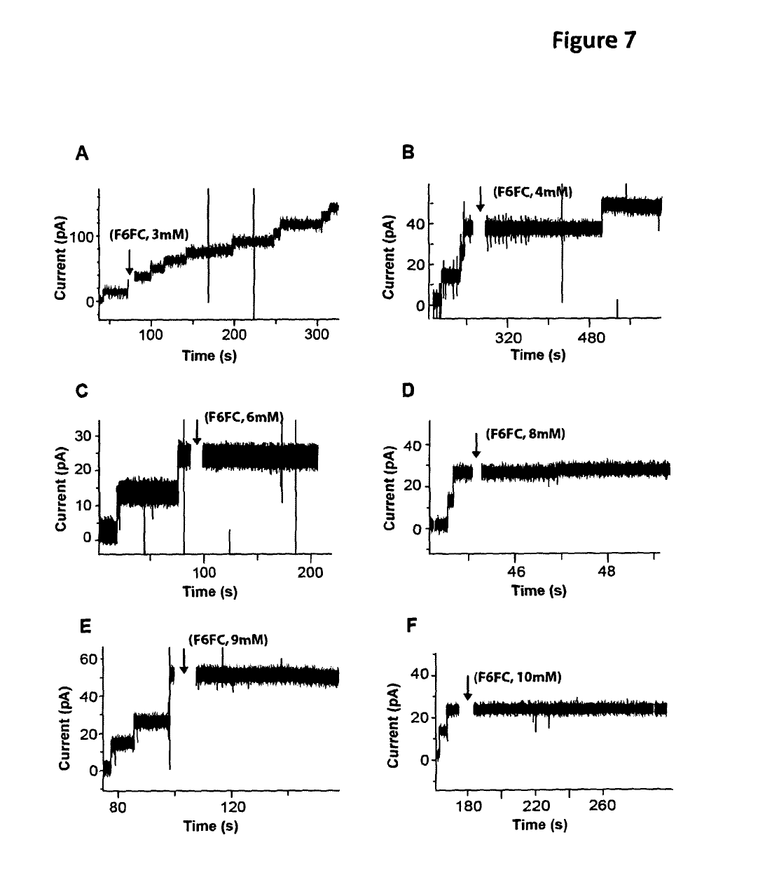

The cessation of membrane protein insertion by F₆FC appears to be effective only at F₆FC concentrations significantly above the CMC. Concentrations of 3 and 4 mM F₆FC, which are just above the reported CMC value of F₆FC (2.2 mM), are not as effective in stopping the membrane insertion of α-HL heptamers (FIG. 7A,B) when compared with higher concentrations of F₆FC, which completely prevent α-HL heptamer insertion (FIG. 7C-F).

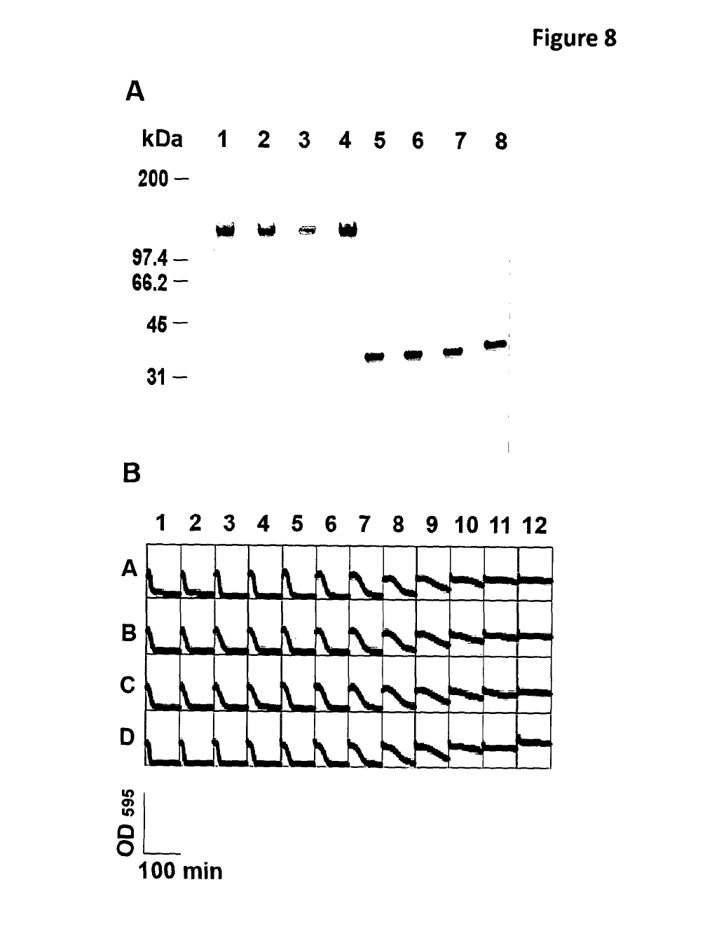

F-amphiphiles might work by denaturing uninserted protein in the aqueous phase. However, SDS-PAGE suggests that the α-HL heptamer does not undergo denaturation after coming into contact with F-amphiphiles, as there is no dissociation to monomers (FIG. 8A). The integrity of the monomer in the presence of or after treatment with the F-amphiphiles at above the CMC was confirmed by a hemolytic assay (FIG. 8B). The ability of α-HL monomers to act on rabbit erythrocytes in conditions under which they will not insert into pure lipid bilayers might be explained by the presence of strong receptors on the red cells that promote irreversible binding and assembly, in competition with sequestration by F-amphiphile aggregates.

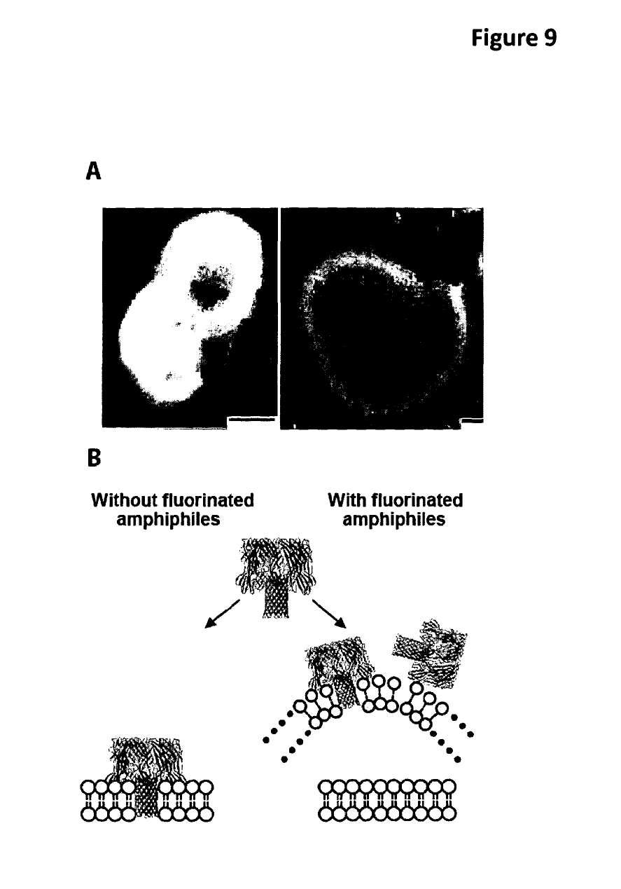

We have proposed sequestration within F-amphiphile aggregates as a plausible mechanism for the arrest of insertion into lipid bilayers of the membrane proteins tested here (FIG. 9). This seems reasonable as proteins have been reported to have a higher affinity for F-amphiphiles over hydrocarbon-based amphiphiles. Under our experimental conditions of low lipid concentration (μM range), the α-HL, MspA and Kcv proteins are suggested to partition quickly onto or into the F-amphiphile aggregates, which are present at higher concentrations (mM monomers), and remain unavailable for insertion into lipid bilayers. Ladokhin and colleagues have carefully examined the effects of F-amphiphiles on the insertion into bilayers of diphtheria toxin T domain and annexin B12 (Rodnin et al., Biophys J 94, 4348-4357). In these cases, insertion was reversible and high concentrations of F-amphiphile removed the proteins from bilayers. The proteins that we have tested bind tightly to bilayers and cannot be removed by F-amphiphiles, although insertion ceases in the presence of these agents, suggesting that the systems are under kinetic rather than thermodynamic control in the cases that we have examined.

### 3 CONCLUSION

Previous reports have shown that F-amphiphiles (e.g. F₆TAC, HFTAC) can assist in the insertion of proteins such as diphtheria toxin and MscL into lipid bilayers by preventing their aggregation in solution (Palchevskyy et al. supra and Park et al. supra). We attempted to extend this work by examining the effects of two commercially available F-amphiphiles, F₆FC and F₆OM, as well as the previously reported F₆TAC, for their effects on the insertion of the α-HL pore, the MspA pore and the Kcv potassium channel into bilayers. We also found that the F-amphiphiles had no effect on the properties of proteins that had already inserted into bilayers. Therefore, F-amphiphile addition might be used to control membrane protein insertion without resorting to methods such as perfusion. The approach might prove useful in single-channel studies of membrane proteins in planar bilayers, and in the manufacture of chips for the rapid screening of membrane proteins or for use as sensor arrays, where one difficulty is to control the number of proteins in each element of the chip.

1. A method for inhibiting the insertion of one or more membrane proteins into a membrane, comprising (a) contacting the proteins and membrane with a fluorinated amphiphile (F-amphiphile) under conditions that in the absence of the F-amphiphile allow the insertion of the proteins into the membrane and (b) thereby inhibiting the insertion of the proteins into the membrane.

2. A method according to claim 1, wherein the one or more membrane proteins are not derived from the mechanosensitive channel of large conductance, MscL, of Escherichia coli.

3. A method according to claim 1, wherein the one or more membrane proteins are derived from a-hemolysin (a-HL), derived from MspA from Mycobacterium smegmatis or derived from Kcv of chlorella virus PBCV-1.

4. A method according to claim 3, wherein the one or more membrane proteins each comprise (a) the sequence shown SEQ ID NO: 2 or a variant thereof, (b) the sequence shown in SEQ ID NO: 14 or a variant thereof or (c) the sequence shown in SEQ ID NO: 16 or a variant thereof.

5-6. (canceled)

7. A method according to claim 1, wherein the F-amphiphile (a) comprises (i) a polar head group and (ii) a hydrophobic tail comprising a fluorinated chain or (b) is F-fos-choline (F₆FC) with a zwitterionic head group, F-octyl maltoside (F₆OM) with a non-ionic disaccharide head group and C₆F₁₃C₂H₄—S-poly[tris(hydroxymethyl)aminomethane] (F₆TAC) with a non-ionic polymeric head group.

8. (canceled)

9. A method according to claim 1, wherein the F-amphiphile is contacted with the proteins and membrane on the cis side.

10. A method according to claim 1, wherein the F-amphiphile is contacted with the proteins and membrane at a concentration greater than the critical micelle concentration (CMC) or at a concentration at least five times greater than the CMC.

11. (canceled)

12. A method according to claim 1, wherein the membrane is a lipid membrane and the lipid-to-protein ratio is lower than 40:1 (w/w) or is 1:1 (w/w).

13. (canceled)

14. Use of a F-amphiphile for inhibiting the insertion of one or more membrane proteins into a membrane.

15. A method for inserting a pre-determined number of membrane proteins into a membrane, comprising (a) contacting more than the pre-determined number of the proteins with the membrane under conditions that allow the insertion of the proteins into the membrane and (b) once the pre-determined number of membrane proteins have inserted in the membrane, contacting the proteins and membrane with a F-amphiphile and thereby inhibiting further insertion of the proteins into the membrane.

16. A method according to claim 15, wherein the method is for inserting a single pore into the membrane.

17. A method according to claim 15, wherein the membrane proteins are derived from a-hemolysin (a-HL), derived from MspA from Mycobacterium smegmatis or derived from Kcv of chlorella virus PBCV-1.

18. A membrane having a predetermined number of membrane proteins inserted therein produced using a method according to claim 15.

19. A method of determining the presence or absence of an analyte, comprising:
(a) contacting the analyte with a membrane according to claim 18, which comprises a transmembrane pore or an ion channel, so that the analyte interacts with the pore or channel; and
(b) measuring the current passing through the pore or channel during the interaction and thereby determining the presence or absence of the analyte.

20. A method according to claim 19, wherein the analyte is an individual nucleotide or is a nucleic acid sequence.

21-22. (canceled)

23. A method of estimating the sequence of a target nucleic acid sequence, comprising:
(a) contacting the target sequence with a membrane according to claim 18, which comprises at least one transmembrane pore so that the target sequence translocates through the pore and a proportion of the nucleotides in the target sequence interacts with the pore; and
(b) measuring the current passing through the pore during each interaction and thereby determining the sequence of the target sequence.

24. A method according to claim 23, wherein the transmembrane pore has a molecular adaptor attached thereto.

25. A method according to claim 23, wherein the transmembrane pore has a nucleic handling enzyme covalently attached thereto and the enzyme controls the translocation of the target sequence through the pore.

26. A kit for inserting a pre-determined number of membrane proteins into a membrane comprising (a) one or more membrane proteins and (b) a fluorinated amphiphile, wherein the membrane proteins are derived from α-hemolysin (α-HL), MspA from Mycobacterium smegmatis or Kcv of chlorella virus PBCV-1.


SEQUENCE LISTING (16 sequences)

SEQ ID NO: 1 - DNA, 882 bp, Staphylococcus aureus
atggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaat

SEQ ID NO: 2 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 3 - DNA, 882 bp, Artificial sequence
  a-HL L135C/N139Q (HL-CQ)
atggcagattctgatattaatattaaaaccggtactacagatattggaagcaatactacagtaaaaacaggtgatttagtcacttatgataaagaaaatggcatgcacaaaaaagtattttatagttttatcgatgataaaaatcacaataaaaaactgctagttattagaacaaaaggtaccattgctggtcaatatagagtttatagcgaagaaggtgctaacaaaagtggtttagcctggccttcagcctttaaggtacagttgcaactacctgataatgaagtagctcaaatatctgattactatccaagaaattcgattgatacaaaagagtatatgagtactttaacttatggattcaacggtaatgttactggtgatgatacaggaaaaattggcggctgtattggtgcacaagtttcgattggtcatacactgaaatatgttcaacctgatttcaaaacaattttagagagcccaactgataaaaaagtaggctggaaagtgatatttaacaatatggtgaatcaaaattggggaccatacgatcgagattcttggaacccggtatatggcaatcaacttttcatgaaaactagaaatggttctatgaaagcagcagataacttccttgatcctaacaaagcaagttctctattatcttcagggttttcaccagacttcgctacagttattactatggatagaaaagcatccaaacaacaaacaaatatagatgtaatatacgaacgagttcgtgatgattaccaattgcattggacttcaacaaattggaaaggtaccaatactaaagataaatggacagatcgttcttcagaaagatataaaatcgattgggaaaaagaagaaatgacaaat

SEQ ID NO: 4 - protein, 293 aa, Artificial sequence
  a-HL L135C/N139Q (HL-CQ)
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGCIGAQVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 5 - DNA, 1390 bp, Escherichia coli
atgatgaacgatggcaaacagcagagcaccttcctgtttcatgattatgaaaccttcggtacccatccggccctggatcgtccggcgcagtttgcggccattcgcaccgatagcgaattcaatgtgattggcgaaccggaagtgttttattgcaaaccggccgatgattatctgccgcagccgggtgcggtgctgattaccggtattaccccgcaggaagcgcgcgcgaaaggtgaaaacgaagcggcgtttgccgcgcgcattcatagcctgtttaccgtgccgaaaacctgcattctgggctataacaatgtgcgcttcgatgatgaagttacccgtaatatcttttatcgtaacttttatgatccgtatgcgtggagctggcagcatgataacagccgttgggatctgctggatgtgatgcgcgcgtgctatgcgctgcgcccggaaggcattaattggccggaaaacgatgatggcctgccgagctttcgtctggaacatctgaccaaagccaacggcattgaacatagcaatgcccatgatgcgatggccgatgtttatgcgaccattgcgatggcgaaactggttaaaacccgtcagccgcgcctgtttgattatctgtttacccaccgtaacaaacacaaactgatggcgctgattgatgttccgcagatgaaaccgctggtgcatgtgagcggcatgtttggcgcctggcgcggcaacaccagctgggtggccccgctggcctggcacccggaaaatcgtaacgccgtgattatggttgatctggccggtgatattagcccgctgctggaactggatagcgataccctgcgtgaacgcctgtataccgccaaaaccgatctgggcgataatgccgccgtgccggtgaaactggttcacattaacaaatgcccggtgctggcccaggcgaacaccctgcgcccggaagatgcggatcgtctgggtattaatcgccagcattgtctggataatctgaaaatcctgcgtgaaaacccgcaggtgcgtgaaaaagtggtggcgatcttcgcggaagcggaaccgttcaccccgagcgataacgtggatgcgcagctgtataacggcttctttagcgatgccgatcgcgcggcgatgaaaatcgttctggaaaccgaaccgcgcaatctgccggcgctggatattacctttgttgataaacgtattgaaaaactgctgtttaattatcgtgcgcgcaattttccgggtaccctggattatgccgaacagcagcgttggctggaacatcgtcgtcaggttttcaccccggaatttctgcagggttatgcggatgaactgcagatgctggttcagcagtatgccgatgataaagaaaaagtggcgctgc

SEQ ID NO: 6 - protein, 485 aa, Escherichia coli
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 7 - DNA, 804 bp, Escherichia coli
atgaaatttgtctcttttaatatcaacggcctgcgcgccagacctcaccagcttgaagccatcgtcgaaaagcaccaaccggatgtgattggcctgcaggagacaaaagttcatgacgatatgtttccgctcgaagaggtggcgaagctcggctacaacgtgttttatcacgggcagaaaggccattatggcgtggcgctgctgaccaaagagacgccgattgccgtgcgtcgcggctttcccggtgacgacgaagaggcgcagcggcggattattatggcggaaatcccctcactgctgggtaatgtcaccgtgatcaacggttacttcccgcagggtgaaagccgcgaccatccgataaaattcccggcaaaagcgcagttttatcagaatctgcaaaactacctggaaaccgaactcaaacgtgataatccggtactgattatgggcgatatgaatatcagccctacagatctggatatcggcattggcgaagaaaaccgtaagcgctggctgcgtaccggtaaatgctctttcctgccggaagagcgcgaatggatggacaggctgatgagctgggggttggtcgataccttccgccatgcgaatccgcaaacagcagatcgtttctcatggtttgattaccgctcaaaaggttttgacgataaccgtggtctgcgcatcgacctgctgctcgccagccaaccgctggcagaatgttgcgtagaaaccggcatcgactatgaaatccgcagcatggaaaaaccgtccgatcacgcccccgtctgggcgaccttccgccgc

SEQ ID NO: 8 - protein, 268 aa, Escherichia coli
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRR

SEQ ID NO: 9 - DNA, 1275 bp, Thermus thermophilus
atgtttcgtcgtaaagaagatctggatccgccgctggcactgctgccgctgaaaggcctgcgcgaagccgccgcactgctggaagaagcgctgcgtcaaggtaaacgcattcgtgttcacggcgactatgatgcggatggcctgaccggcaccgcgatcctggttcgtggtctggccgccctgggtgcggatgttcatccgtttatcccgcaccgcctggaagaaggctatggtgtcctgatggaacgcgtcccggaacatctggaagcctcggacctgtttctgaccgttgactgcggcattaccaaccatgcggaactgcgcgaactgctggaaaatggcgtggaagtcattgttaccgatcatcatacgccgggcaaaacgccgccgccgggtctggtcgtgcatccggcgctgacgccggatctgaaagaaaaaccgaccggcgcaggcgtggcgtttctgctgctgtgggcactgcatgaacgcctgggcctgccgccgccgctggaatacgcggacctggcagccgttggcaccattgccgacgttgccccgctgtggggttggaatcgtgcactggtgaaagaaggtctggcacgcatcccggcttcatcttgggtgggcctgcgtctgctggctgaagccgtgggctataccggcaaagcggtcgaagtcgctttccgcatcgcgccgcgcatcaatgcggcttcccgcctgggcgaagcggaaaaagccctgcgcctgctgctgacggatgatgcggcagaagctcaggcgctggtcggcgaactgcaccgtctgaacgcccgtcgtcagaccctggaagaagcgatgctgcgcaaactgctgccgcaggccgacccggaagcgaaagccatcgttctgctggacccggaaggccatccgggtgttatgggtattgtggcctctcgcatcctggaagcgaccctgcgcccggtctttctggtggcccagggcaaaggcaccgtgcgttcgctggctccgatttccgccgtcgaagcactgcgcagcgcggaagatctgctgctgcgttatggtggtcataaagaagcggcgggtttcgcaatggatgaagcgctgtttccggcgttcaaagcacgcgttgaagcgtatgccgcacgtttcccggatccggttcgtgaagtggcactgctggatctgctgccggaaccgggcctgctgccgcaggtgttccgtgaactggcactgctggaaccgtatggtgaaggtaacccggaaccgctgttcctg

SEQ ID NO: 10 - protein, 425 aa, Thermus thermophilus
MFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPPPGLVVHPALTPDLKEKPTGAGVAFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFL

SEQ ID NO: 11 - DNA, 738 bp, Coliphage lambda
tccggaagcggctctggtagtggttctggcatgacaccggacattatcctgcagcgtaccgggatcgatgtgagagctgtcgaacagggggatgatgcgtggcacaaattacggctcggcgtcatcaccgcttcagaagttcacaacgtgatagcaaaaccccgctccggaaagaagtggcctgacatgaaaatgtcctacttccacaccctgcttgctgaggtttgcaccggtgtggctccggaagttaacgctaaagcactggcctggggaaaacagtacgagaacgacgccagaaccctgtttgaattcacttccggcgtgaatgttactgaatccccgatcatctatcgcgacgaaagtatgcgtaccgcctgctctcccgatggtttatgcagtgacggcaacggccttgaactgaaatgcccgtttacctcccgggatttcatgaagttccggctcggtggtttcgaggccataaagtcagcttacatggcccaggtgcagtacagcatgtgggtgacgcgaaaaaatgcctggtactttgccaactatgacccgcgtatgaagcgtgaaggcctgcattatgtcgtgattgagcgggatgaaaagtacatggcgagttttgacgagatcgtgccggagttcatcgaaaaaatggacgaggcactggctgaaattggttttgtatttggggagcaatggcgatctggctctggttccggcagcggttccgga

SEQ ID NO: 12 - protein, 226 aa, Coliphage lambda
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 13 - DNA, 700 bp, Mycobacterium smegmatis
ggggccgccggcgatacagttagggagaacatgaaggcaatcagtcgggtgctgatcgcgatggttgcagccatcgcggcgcttttcacgagcacaggcacctctcacgcaggcctggacaacgagctgagcctcgttgatggccaggaccgcaccctcaccgtgcagcagtgggacaccttcctcaatggtgtgttccccctggaccgcaaccgtcttacccgtgagtggttccactccggtcgcgccaagtacatcgtggccggccccggtgccgacgagttcgagggcacgctggaactcggctaccagatcggcttcccgtggtcgctgggtgtgggcatcaacttcagctacaccaccccgaacatcctgatcgacgacggtgacatcaccgctccgccgttcggcctgaactcggtcatcaccccgaacctgttccccggtgtgtcgatctcggcagatctgggcaacggccccggcatccaggaagtcgcaacgttctcggtcgacgtctccggcgccgagggtggcgtggccgtgtcgaacgcccacggcaccgtgaccggtgcggccggcggtgtgctgctgcgtccgttcgcccgcctgatcgcctcgaccggtgactcggtcaccacctacggcgaaccctggaacatgaactgattcctggaccgccgttcggtcgctgagaccgctt

SEQ ID NO: 14 - protein, 209 aa, Mycobacterium smegmatis
MKAISRVLIAMVAAIAALFTSTGTSHAGLDNELSLVDGQDRTLTVQQWDTFLNGVFPLDRNRLTREWFHSGRAKYIVAGPGADEFEGTLEYQIGFPWSLGVGINFSYTTPNILIDDGDITAPPFGLNSVITPNLFPGVSISADLGNGPGIQEVATFSVDVSGAEGGVAVSNAHGTVTGAAGGVLLRPFARLIASTGDSVTTYGEPWNMN

SEQ ID NO: 15 - DNA, 284 bp, Paramecium bursaria Chlorella virus 1
  misc_feature (254)..(254): n is a, c, g, or t
ttagtgttttctaaattcctgacccgcacggaaccatttatgatccacttattcatcttagcgatgtttgtgatgatttacaagttctttccgggtggctttgagaacaacttctctgtggcgaatccggacaaaaaagcgtcttggattgattgtatttattttggtgtgaccacccactctaccgttggtttcggtgatatcttaccaaaaacgacgggcgcgaaactgtgcacgattgcccacattgtgancggtgttctttattgttttaanccctgtga

SEQ ID NO: 16 - protein, 96 aa, Paramecium bursaria Chlorella virus 1
  MISC_FEATURE (86)..(86): Xaa is Asn, Ser, Thr or Ile
MLVFSKFLTRTEPFMIHLFILAMFVMIYKFFPGGFENNFSVANPDKKASWIDCIYFGVTTHSTVGFGDILPKTTGAKLCTIAHIVXGVLYCFXPCD